SEQUENCE LISTING (32 sequences)

SEQ ID NO: 1 - protein, 194 aa, Artificial Sequence
  Synthetic
  MISC_FEATURE (16)..(16): X is R or K
  MISC_FEATURE (37)..(37): X is V or F
  MISC_FEATURE (73)..(73): X is R or K
  MISC_FEATURE (85)..(85): X is T or A
  MISC_FEATURE (87)..(87): X is K, N, R, or T
  MISC_FEATURE (109)..(109): X is K or N
  MISC_FEATURE (115)..(115): X is V, I, or A
  MISC_FEATURE (116)..(116): X is V, G, or S
  MISC_FEATURE (119)..(119): X is Y, H, or S
  MISC_FEATURE (122)..(122): X is L, Q, or R
  MISC_FEATURE (123)..(123): X is Y or F
  MISC_FEATURE (125)..(126): X is L or F
  MISC_FEATURE (128)..(128): X is K or N
  MISC_FEATURE (140)..(140): X is T or N
  MISC_FEATURE (145)..(145): X is A, V or S
  MISC_FEATURE (146)..(146): X is A, G or S
  MISC_FEATURE (147)..(147): X is S, G, I, N, or R
  MISC_FEATURE (148)..(148): X is T, A, I, K, or R
  MISC_FEATURE (153)..(153): X is V or A
  MISC_FEATURE (154)..(154): X is E, A, D, K, L, or Q
  MISC_FEATURE (155)..(155): X is G, K, or R
  MISC_FEATURE (156)..(156): X is F or I
  MISC_FEATURE (160)..(160): X is F, L or S
  MISC_FEATURE (163)..(163): X is K, Q,  L or R
  MISC_FEATURE (164)..(164): X is S, L or P
  MISC_FEATURE (166)..(166): X is G or S
  MISC_FEATURE (171)..(171): X is N, T, or Y
  MISC_FEATURE (173)..(173): X is V, F, or I
  MISC_FEATURE (174)..(174): X is G or D
  MISC_FEATURE (175)..(175): X is Y, H, or W
NITNLCPFGEVFNATXFASVYAWNRKRISNCVADYSXLYNSASFSTFKCYGVSPTKLNDLCFTNVYADSFVIXGDEVRQIAPGQXGXIADYNYKLPDDFTGCVIAWNSXNLDSKXXGNXNYXXRXXRXSNLKPFERDISXEIYQXXXXPCNGXXXXNCYXPLXXYXFQPTXGXXXQPYRVVVLSFELLHAPATV

SEQ ID NO: 2 - protein, 194 aa, Artificial Sequence
  Synthetic
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 3 - protein, 154 aa, Artificial Sequence
  Synthetic
  MISC_FEATURE (5)..(5): X is E or C
  MISC_FEATURE (8)..(8): X is L or C
  MISC_FEATURE (9)..(9): X is T or G
  MISC_FEATURE (22)..(22): X is F or A
  MISC_FEATURE (40)..(40): X is R or C
  MISC_FEATURE (41)..(41): X is H, F, or M
  MISC_FEATURE (52)..(52): X is R or C
  MISC_FEATURE (54)..(54): X is P or C
  MISC_FEATURE (61)..(61): X is V or C
  MISC_FEATURE (82)..(82): X is I or C
  MISC_FEATURE (86)..(86): X is T or C
  MISC_FEATURE (87)..(87): X is P or G
  MISC_FEATURE (88)..(88): X is H or S
  MISC_FEATURE (105)..(105): X is L or C
  MISC_FEATURE (119)..(119): X is D or C
  MISC_FEATURE (127)..(127): X is R or A
  MISC_FEATURE (131)..(131): X is K or C
  MISC_FEATURE (142)..(142): X is S or C
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLR

SEQ ID NO: 4 - protein, 154 aa, Artificial Sequence
  Synthetic
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLR

SEQ ID NO: 5 - protein, 354 aa, Artificial Sequence
  Synthetic
  MISC_FEATURE (5)..(5): X is E or C
  MISC_FEATURE (8)..(8): X is L or C
  MISC_FEATURE (9)..(9): X is Tor G
  MISC_FEATURE (22)..(22): X is F or A
  MISC_FEATURE (40)..(40): X is R or C
  MISC_FEATURE (41)..(41): X is H, F, or M
  MISC_FEATURE (52)..(52): X is R or C
  MISC_FEATURE (54)..(54): X is P or C
  MISC_FEATURE (61)..(61): X is V or C
  MISC_FEATURE (82)..(82): X is I or C
  MISC_FEATURE (86)..(86): X is T or C
  MISC_FEATURE (87)..(87): X is P or G
  MISC_FEATURE (88)..(88): X is H or S
  MISC_FEATURE (105)..(105): X is L or C
  MISC_FEATURE (119)..(119): X is D or C
  MISC_FEATURE (127)..(127): X is R or A
  MISC_FEATURE (131)..(131): X is K or C
  MISC_FEATURE (142)..(142): X is S or C
  MISC_FEATURE (155)..(157): can be reapted “n” times
  MISC_FEATURE (233)..(233): X is R or K
  MISC_FEATURE (247)..(247): X is K or N
  MISC_FEATURE (269)..(269): X is K or N
  MISC_FEATURE (275)..(275): X is V, I, or A
  MISC_FEATURE (276)..(276): X is V, G, or S
  MISC_FEATURE (283)..(283): X is Y or F
  MISC_FEATURE (285)..(285): X is L or F
  MISC_FEATURE (286)..(286): X is L or F
  MISC_FEATURE (305)..(305): X is A or V
  MISC_FEATURE (306)..(306): X is G, S or A
  MISC_FEATURE (307)..(307): X is S, N, I, G, or R
  MISC_FEATURE (308)..(308): X is T, I, A,  or K
  MISC_FEATURE (314)..(314): X is E, Q, K, A, L, or D
  MISC_FEATURE (315)..(315): X is G, R, or K
  MISC_FEATURE (320)..(320): X is F, S, or L
  MISC_FEATURE (323)..(323): X is L or Q
  MISC_FEATURE (324)..(324): X is S, L, or P
  MISC_FEATURE (331)..(331): X is N or Y
  MISC_FEATURE (334)..(334): X is G or D
  MISC_FEATURE (335)..(335): X is Y or W
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGGSGGGNITNLCPFGEVFNATRFASVYAWNRKRISNCVADYSVLYNSASFSTFKCYGVSPTKLNDLCFTNVYADSFVIXGDEVRQIAPGQTGXIADYNYKLPDDFTGCVIAWNSXNLDSKXXGNYNYLXRXXRKSNLKPFERDISTEIYQXXXXPCNGVXXFNCYXPLXXYGFQPTXGVXXQPYRVVVLSFELLHAPATV

SEQ ID NO: 6 - protein, 354 aa, Artificial Sequence
  Synthetic
  MISC_FEATURE (5)..(5): X is E or C
  MISC_FEATURE (8)..(8): X is L or C
  MISC_FEATURE (9)..(9): X is T or G
  MISC_FEATURE (22)..(22): X is F or A
  MISC_FEATURE (40)..(40): X is R or C
  MISC_FEATURE (41)..(41): X is H, F, or M
  MISC_FEATURE (52)..(52): X is R or C
  MISC_FEATURE (54)..(54): X is P or C
  MISC_FEATURE (61)..(61): X is V or C
  MISC_FEATURE (82)..(82): X is I or C
  MISC_FEATURE (86)..(86): X is T or C
  MISC_FEATURE (87)..(87): X is P or G
  MISC_FEATURE (88)..(88): X is H or S
  MISC_FEATURE (105)..(105): X is L or C
  MISC_FEATURE (119)..(119): X is D or C
  MISC_FEATURE (127)..(127): X is R or A
  MISC_FEATURE (131)..(131): X is K or C
  MISC_FEATURE (142)..(142): X is S or C
  MISC_FEATURE (155)..(157): is optionally repeated “n” times
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGGSGGGNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 7 - protein, 363 aa, Artificial Sequence
  Synthetic
  MISC_FEATURE (242)..(242): X is R or K
  MISC_FEATURE (256)..(256): X is N or K
  MISC_FEATURE (278)..(278): X is N or K
  MISC_FEATURE (284)..(284): X is V, I, or A
  MISC_FEATURE (285)..(285): X is V, G, or S
  MISC_FEATURE (292)..(292): X is Y or F
  MISC_FEATURE (294)..(295): X is L or F
  MISC_FEATURE (314)..(314): X is A or V
  MISC_FEATURE (315)..(315): X is G, A, or S
  MISC_FEATURE (316)..(316): X is S, N, I, G, or R
  MISC_FEATURE (317)..(317): X is T, A, I, or K
  MISC_FEATURE (323)..(323): X is E, Q, K, A, L, or D
  MISC_FEATURE (324)..(324): X is G, R, or K
  MISC_FEATURE (329)..(329): X is F, S, or L
  MISC_FEATURE (332)..(332): X is Q or L
  MISC_FEATURE (333)..(333): X is S, P or L
  MISC_FEATURE (340)..(340): X is N or Y
  MISC_FEATURE (343)..(343): X is G or D
  MISC_FEATURE (344)..(344): X is Y or W
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGNITNLCPFGEVFNATRFASVYAWNRKRISNCVADYSVLYNSASFSTFKCYGVSPTKLNDLCFTNVYADSFVIXGDEVRQIAPGQTGXIADYNYKLPDDFTGCVIAWNSXNLDSKXXGNYNYLXRXXRKSNLKPFERDISTEIYQXXXXPCNGVXXFNCYXPLXXYGFQPTXGVXXQPYRVVVLSFELLHAPATV

SEQ ID NO: 8 - protein, 373 aa, Artificial Sequence
  Synthetic
  MISC_FEATURE (245)..(245): X is R or K
  MISC_FEATURE (259)..(259): X is N or K
  MISC_FEATURE (281)..(281): X is N or K
  MISC_FEATURE (287)..(287): X is V, I or A
  MISC_FEATURE (288)..(288): X is V, G,  or S
  MISC_FEATURE (295)..(295): X is Y or F
  MISC_FEATURE (297)..(298): X is L or F
  MISC_FEATURE (317)..(317): X is A or V
  MISC_FEATURE (318)..(318): X is G, A, or S
  MISC_FEATURE (319)..(319): X is S, N, I, G, or R
  MISC_FEATURE (320)..(320): X is T, I, A, or K
  MISC_FEATURE (326)..(326): X is E, Q, K, A, L, or D
  MISC_FEATURE (327)..(327): X is G, R, or K
  MISC_FEATURE (332)..(332): X is F, S, or L
  MISC_FEATURE (335)..(335): X is Q or L
  MISC_FEATURE (336)..(336): X is S, P, or L
  MISC_FEATURE (343)..(343): X is N or Y
  MISC_FEATURE (343)..(343): X is N or Y
  MISC_FEATURE (346)..(346): X is G or D
  MISC_FEATURE (347)..(347): X is Y or W
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFGEVFNATRFASVYAWNRKRISNCVADYSVLYNSASFSTFKCYGVSPTKLNDLCFTNVYADSFVIXGDEVRQIAPGQTGXIADYNYKLPDDFTGCVIAWNSXNLDSKXXGNYNYLXRXXRKSNLKPFERDISTEIYQXXXXPCNGVXXFNCYXPLXXYGFQPTXGVXXQPYRVVVLSFELLHAPATVCGPKKST

SEQ ID NO: 9 - protein, 363 aa, Artificial Sequence
  Synthetic
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 10 - protein, 373 aa, Artificial Sequence
  Synthetic
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 11 - protein, 194 aa, Artificial Sequence
  Synthetic
  MISC_FEATURE (73)..(73): X is R or K
  MISC_FEATURE (87)..(87): X is N or K
  MISC_FEATURE (109)..(109): X is N or K
  MISC_FEATURE (115)..(115): X is V, I or A
  MISC_FEATURE (116)..(116): X is V, G or S
  MISC_FEATURE (123)..(123): X is Y or F
  MISC_FEATURE (125)..(126): X is L or F
  MISC_FEATURE (145)..(145): X is A or V
  MISC_FEATURE (146)..(146): X is G, S, or A
  MISC_FEATURE (147)..(147): X is S, N, I, G, or R
  MISC_FEATURE (148)..(148): X is T, I, A, or K
  MISC_FEATURE (154)..(154): X is E, Q, K, A, L, or D
  MISC_FEATURE (155)..(155): X is G, R, or K
  MISC_FEATURE (160)..(160): X is F, S, or L
  MISC_FEATURE (163)..(163): X is Q or L
  MISC_FEATURE (164)..(164): X is S, P or L
  MISC_FEATURE (171)..(171): X is N or Y
  MISC_FEATURE (174)..(174): X is G or D
  MISC_FEATURE (175)..(175): X is Y or W
NITNLCPFGEVFNATRFASVYAWNRKRISNCVADYSVLYNSASFSTFKCYGVSPTKLNDLCFTNVYADSFVIXGDEVRQIAPGQTGXIADYNYKLPDDFTGCVIAWNSXNLDSKXXGNYNYLXRXXRKSNLKPFERDISTEIYQXXXXPCNGVXXFNCYXPLXXYGFQPTXGVXXQPYRVVVLSFELLHAPATV

SEQ ID NO: 12 - RNA, 17 nt, Artificial Sequence
  Synthetic
gggagacugccaccaug

SEQ ID NO: 13 - RNA, 17 nt, Artificial Sequence
  Synthetic
gggagacugccaagaug

SEQ ID NO: 14 - DNA, 17 bp, Artificial Sequence
  Synthetic
taatacgactcactata

SEQ ID NO: 15 - skipped

SEQ ID NO: 16 - RNA, 17 nt, Artificial Sequence
  Synthetic
  misc_feature (13)..(13): n is C or A
  misc_feature (14)..(14): n is C or G
gggagacugccannaug

SEQ ID NO: 17 - skipped

SEQ ID NO: 18 - RNA, 132 nt, Artificial Sequence
  Synthetic
gcucgcuuucuugcuguccaauuucuauuaaagguuccuuuguucccuaaguccaacuacuaaacugggggauauuaugaagggccuugagcaucuggauucugccuaauaaaaaacauuuauuuucauugc

SEQ ID NO: 19-21 - skipped (3 entries)

SEQ ID NO: 22 - protein, 21 aa, Artificial Sequence
  Synthetic
MMYRMQLLSCIALSLALVTNS

SEQ ID NO: 23 - protein, 20 aa, Artificial Sequence
  Synthetic
MYRMQLLSCIALSLALVTNS

SEQ ID NO: 24 - protein, 354 aa, Artificial Sequence
  Synthetic
  MISC_FEATURE (5)..(5): X is E or C
  MISC_FEATURE (8)..(8): X is L or C
  MISC_FEATURE (9)..(9): X is T or G
  MISC_FEATURE (22)..(22): X is F or A
  MISC_FEATURE (40)..(40): X is R or C
  MISC_FEATURE (41)..(41): X is H, F, or M
  MISC_FEATURE (52)..(52): X is R or C
  MISC_FEATURE (54)..(54): X is P or C
  MISC_FEATURE (61)..(61): X is V or C
  MISC_FEATURE (82)..(82): X is I or C
  MISC_FEATURE (86)..(86): X is T or C
  MISC_FEATURE (87)..(87): X is P or G
  MISC_FEATURE (88)..(88): X is H or S
  MISC_FEATURE (105)..(105): X is L or C
  MISC_FEATURE (119)..(119): X is D or C
  MISC_FEATURE (127)..(127): X is R or A
  MISC_FEATURE (131)..(131): X is K or C
  MISC_FEATURE (142)..(142): X is S or C
  MISC_FEATURE (155)..(157): can be repeated “n” times
  MISC_FEATURE (176)..(176): X is R or K
  MISC_FEATURE (197)..(197): X is V or F
  MISC_FEATURE (233)..(233): X is R or K
  MISC_FEATURE (245)..(245): X is T or A
  MISC_FEATURE (247)..(247): X is K, N, T or R
  MISC_FEATURE (269)..(269): X is K or N
  MISC_FEATURE (275)..(275): X is V, I or A
  MISC_FEATURE (276)..(276): X is V, G or S
  MISC_FEATURE (279)..(279): X is Y, H or S
  MISC_FEATURE (282)..(282): X is L, Q or R
  MISC_FEATURE (283)..(283): X is Y or F
  MISC_FEATURE (285)..(285): X is L or F
  MISC_FEATURE (286)..(286): X is L or F
  MISC_FEATURE (288)..(288): X is K or N
  MISC_FEATURE (300)..(300): X is T or N
  MISC_FEATURE (305)..(305): X is A, S or V
  MISC_FEATURE (306)..(306): X is A, S or V
  MISC_FEATURE (307)..(307): X is A, S or G
  MISC_FEATURE (307)..(307): X is S, G, I, N, or R
  MISC_FEATURE (308)..(308): X is T, A, I, K, or R
  MISC_FEATURE (313)..(313): X is V or A
  MISC_FEATURE (314)..(314): X is E, A, D, K, L or Q
  MISC_FEATURE (315)..(315): X is G, K or R
  MISC_FEATURE (316)..(316): X is F or I
  MISC_FEATURE (320)..(320): X is F, L or S
  MISC_FEATURE (323)..(323): X is Q, K, L, or  R
  MISC_FEATURE (324)..(324): X is S, L, or P
  MISC_FEATURE (326)..(326): X is S or G
  MISC_FEATURE (331)..(331): X is N, T or Y
  MISC_FEATURE (333)..(333): X is V, F, or I
  MISC_FEATURE (334)..(334): X is G or D
  MISC_FEATURE (335)..(335): X is Y, H, or W
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGGSGGGNITNLCPFGEVFNATXFASVYAWNRKRISNCVADYSXLYNSASFSTFKCYGVSPTKLNDLCFTNVYADSFVIXGDEVRQIAPGQXGXIADYNYKLPDDFTGCVIAWNSXNLDSKXXGNXNYXXRXXRXSNLKPFERDISXEIYQXXXXPCNGXXXXNCYXPLXXYXFQPTXGXXXQPYRVVVLSFELLHAPATV

SEQ ID NO: 25 - protein, 363 aa, Artificial Sequence
  Synthetic
  MISC_FEATURE (185)..(185): X is R or K
  MISC_FEATURE (206)..(206): X is V or F
  MISC_FEATURE (242)..(242): X is R or K
  MISC_FEATURE (254)..(254): X is T or A
  MISC_FEATURE (256)..(256): X is K, N, R or T
  MISC_FEATURE (278)..(278): X is N or K
  MISC_FEATURE (284)..(284): X is V, I, or A
  MISC_FEATURE (285)..(285): X is V, G, or S
  MISC_FEATURE (288)..(288): X is Y, H, or S
  MISC_FEATURE (291)..(291): X is L, Q, or R
  MISC_FEATURE (292)..(292): X is Y or F
  MISC_FEATURE (294)..(295): X is L or F
  MISC_FEATURE (297)..(297): X is K or N
  MISC_FEATURE (309)..(309): X is T or N
  MISC_FEATURE (314)..(314): X is A, S, or V
  MISC_FEATURE (315)..(315): X is A, S, or G
  MISC_FEATURE (316)..(316): X is S, G, I, N, or R
  MISC_FEATURE (317)..(317): X is T, A, I, K, or R
  MISC_FEATURE (322)..(322): X is V or A
  MISC_FEATURE (323)..(323): X is E, D, K, L, Q or A
  MISC_FEATURE (324)..(324): X is G, K, or R
  MISC_FEATURE (325)..(325): X is F or I
  MISC_FEATURE (329)..(329): X is F, L, or S
  MISC_FEATURE (332)..(332): X is Q, K, L, or R
  MISC_FEATURE (333)..(333): X is S, L, or P
  MISC_FEATURE (335)..(335): X is S or G
  MISC_FEATURE (340)..(340): X is N, T, or Y
  MISC_FEATURE (342)..(342): X is V, F, or I
  MISC_FEATURE (343)..(343): X is G or D
  MISC_FEATURE (344)..(344): X is Y, H,  or W
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGNITNLCPFGEVFNATXFASVYAWNRKRISNCVADYSXLYNSASFSTFKCYGVSPTKLNDLCFTNVYADSFVIXGDEVRQIAPGQXGXIADYNYKLPDDFTGCVIAWNSXNLDSKXXGNXNYXXRXXRXSNLKPFERDISXEIYQXXXXPCNGXXXXNCYXPLXXYXFQPTXGXXXQPYRVVVLSFELLHAPATV

SEQ ID NO: 26 - protein, 373 aa, Artificial Sequence
  Synthetic
  MISC_FEATURE (188)..(188): X is R or K
  MISC_FEATURE (209)..(209): X is V or F
  MISC_FEATURE (245)..(245): X is R or K
  MISC_FEATURE (257)..(257): X is T or A
  MISC_FEATURE (259)..(259): X is K, N, R or T
  MISC_FEATURE (281)..(281): X is K or N
  MISC_FEATURE (287)..(287): X is V, A, or I
  MISC_FEATURE (288)..(288): X is V, G, or S
  MISC_FEATURE (291)..(291): X is Y, H, or S
  MISC_FEATURE (294)..(294): X is L, Q or R
  MISC_FEATURE (295)..(295): X is Y or F
  MISC_FEATURE (297)..(298): X is L or F
  MISC_FEATURE (300)..(300): X is K or N
  MISC_FEATURE (312)..(312): X is T or N
  MISC_FEATURE (317)..(317): X is A, S, or V
  MISC_FEATURE (318)..(318): X is A, S, or G
  MISC_FEATURE (319)..(319): X is S, G, I, N,  or R
  MISC_FEATURE (320)..(320): X is T, A, I, K, or R
  MISC_FEATURE (320)..(320): X is T, A, I, K, or R
  MISC_FEATURE (325)..(325): X is V or A
  MISC_FEATURE (326)..(326): X is E, A, D, K, L, or Q
  MISC_FEATURE (327)..(327): X is G, K or R
  MISC_FEATURE (328)..(328): X is F or I
  MISC_FEATURE (332)..(332): X is F, L or S
  MISC_FEATURE (335)..(335): X is Q, K, L, or R
  MISC_FEATURE (336)..(336): X is S, L, or P
  MISC_FEATURE (338)..(338): X is S or G
  MISC_FEATURE (343)..(343): X is N, T or Y
  MISC_FEATURE (345)..(345): X is V, F, or Y
  MISC_FEATURE (346)..(346): X is G or D
  MISC_FEATURE (347)..(347): X is Y, H or W
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFGEVFNATXFASVYAWNRKRISNCVADYSXLYNSASFSTFKCYGVSPTKLNDLCFTNVYADSFVIXGDEVRQIAPGQXGXIADYNYKLPDDFTGCVIAWNSXNLDSKXXGNXNYXXRXXRXSNLKPFERDISXEIYQXXXXPCNGXXXXNCYXPLXXYXFQPTXGXXXQPYRVVVLSFELLHAPATVCGPKKST

SEQ ID NO: 27 - protein, 363 aa, Artificial Sequence
  Synthetic
  MISC_FEATURE (185)..(185): X is R or K
  MISC_FEATURE (206)..(206): X is V or F
  MISC_FEATURE (256)..(256): X is K, N, or T
  MISC_FEATURE (291)..(291): X is L, Q, or R
  MISC_FEATURE (317)..(317): X is T, K, or R
  MISC_FEATURE (323)..(323): X is E, K, or Q
  MISC_FEATURE (329)..(329): X is F or S
  MISC_FEATURE (340)..(340): X is N or Y
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGNITNLCPFGEVFNATXFASVYAWNRKRISNCVADYSXLYNSASFSTFKCYGVSPTKLNDLCFTNVYADSFVIRGDEVRQIAPGQTGXIADYNYKLPDDFTGCVIAWNSNNLDSKVGGNYNYXYRLFRKSNLKPFERDISTEIYQAGSXPCNGVXGFNCYXPLQSYGFQPTXGVGYQPYRVVVLSFELLHAPATV

SEQ ID NO: 28 - protein, 373 aa, Artificial Sequence
  Synthetic
  MISC_FEATURE (188)..(188): X is R or K
  MISC_FEATURE (209)..(209): X is V or F
  MISC_FEATURE (259)..(259): X is K, N or T
  MISC_FEATURE (259)..(259): X is K, N or T
  MISC_FEATURE (294)..(294): X is L, Q or R
  MISC_FEATURE (320)..(320): X is T, K, or R
  MISC_FEATURE (326)..(326): X is E, K, or Q
  MISC_FEATURE (332)..(332): X is F or S
  MISC_FEATURE (343)..(343): X is N or Y
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFGEVFNATXFASVYAWNRKRISNCVADYSXLYNSASFSTFKCYGVSPTKLNDLCFTNVYADSFVIRGDEVRQIAPGQTGXIADYNYKLPDDFTGCVIAWNSNNLDSKVGGNYNYXYRLFRKSNLKPFERDISTEIYQAGSXPCNGVXGFNCYXPLQSYGFQPTXGVGYQPYRVVVLSFELLHAPATVCGPKKST

SEQ ID NO: 29 - protein, 373 aa, Artificial Sequence
  Synthetic
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKST

SEQ ID NO: 30 - protein, 373 aa, Artificial Sequence
  Synthetic
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKST

SEQ ID NO: 31 - protein, 373 aa, Artificial Sequence
  Synthetic
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKST

SEQ ID NO: 32 - protein, 373 aa, Artificial Sequence
  Synthetic
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKST

(19) United States
(12) Patent Application Publication
(10) Pub. No.: US 2023/0322863 A1
(43) Pub. Date: Oct. 12, 2023

(54) REAGENTS AND METHODS FOR PREVENTING, TREATING OR LIMITING SEVERE ACUTE RESPIRATORY SYNDROME (SARS) CORONAVIRUS INFECTION

(71) Applicant: PHYLEX BIOSCIENCES, INC., Del Mar, CA (US)

(72) Inventor: Pascal BRANDYS, Del Mar, CA (US)

(21) Appl. No.: 18/042,083

(22) PCT Filed: Aug. 23, 2021
(86) PCT No.: PCT/US2021/047053

§ 371 Date: Feb. 17, 2023

Related U.S. Application Data
(60) Provisional application No. 63/069,573, filed on Aug. 24, 2020.

(51) Int. Cl. C07K 14/005; C12N 15/86; G01N 33/569; A61P 31/14; A61K 39/215

(52) U.S. Cl. CPC C07K 14/005; C12N 15/86; G01N 33/56983; A61P 31/14; A61K 39/215; G01N 2333/165; G01N 2469/20; C12N 2770/20034; C12N 2770/20071; C12N 2770/20043; C12N 2770/20022; A61K 2039/53

(57) ABSTRACT

The present disclosure provides polypeptides, and nucleic acids encoding the polypeptides, that include severe acute respiratory syndrome Co-V-2 (SARS-CoV-2) spike polypeptide receptor-binding domain (RBD) polypeptides or variants thereof, which are capable of multimerization and thus presenting multiple copies of the RBD to enhance the immune response generated when the polypeptide is administered to a subject. The disclosure also provides multimers, scaffolds, compositions, pharmaceutical compositions, and vaccines that include the polypeptides and/or nucleic acids that encode such polypeptides.

### CROSS REFERENCE

This application claims priority to U.S. Provisional Patent Application Ser. No. 63/069,573 filed Aug. 24, 2020 incorporated by reference herein in its entirety,

### SEQUENCE LISTING STATEMENT

A computer readable form of the Sequence Listing is filed with this application by electronic submission and is incorporated into this application by reference in its entirety. The Sequence Listing is contained in the file created on Aug. 19, 2021 having the file name “20-1279-WO-SeqList_ST25.txt” and is 79 kb in size.

### BACKGROUND

Three highly, pathogenic human coronaviruses (CoVs) have been identified to date: severe acute respiratory syndrome (SARS) coronavirus (SARS-CoV), Middle East respiratory syndrome coronavirus (MFRS-CoV) and a 2019 novel coronavirus (2019-nCoV), as previously termed by the World Health Organization (WHO).

The 2019-nCoV writs first reported in Wuhan, China in December 2010 from patients with pneumonia, and it has far exceeded both SARS-CoV and MERS-CoV in its rate of transmission among humans. 2019-nCoV was renamed SARS-CoV-2 by Coronaviridae Study Group (CSG) of the Internatiottal Committee on Taxonomy of Viruses (ICTV). The disease and the virus causing it were named Coronavirus Disease 2019 (COVID-19) and the COVID-19 virus, respecetively, by the WHO. As of Aug. 13, 2021, more than 205 million cases of COVID-19 were reported, resulting in more than 4.3 million reported deaths, in at least 200 countries and territories.

SARS-CoV-2 is a single, non-segment and positive-stranded RNA virus with envelope. Its genomic RNA consists of 29,903 nucleotides, two thirds of its 5′-encoding nonstructural RNA replicase polyprotein and one third of its 3′-encoding structural proteins, including spike (S), envelope (E), membrane (M), and nucleocapsid (N) proteins.

The SARS-C6V-2 S protein is a type 1 transmembrane envelope glycoprotein and consists of an S1 surface subunit, which is responsible for receptor binding, and an S2 transmembrane subunit, which mediates membrane fusion.

The S protein mediates viral entry into host cells by first binding to a host receptor through the receptor-binding domain (RED) in the S1 subunit and then fusing the viral and host membranes through the S2 subunit. The entry of SARS-CoV-2 is it by binding of the S protein to the cellular receptor angiotensin-converting enzyme 2 (ACE2).

In SARS-CoV-2 a fragment of 194 residues spanning the residues 331-524 in the S1 subunit is the minimal reference RBD used in this disclosure. Alternatively, a fragment of 204 residues spanning the residues 328-531 in the S1 subunit comprising the minimal RBD is also used in this disclosure.

### SUMMARY

In a first aspect, the disclosure provides isolated poly-peptides comprising:

(a) a receptor binding domain (RBD) comprising an amino acid sequence at least 70%, 75%, 80%, 85%, 90%, 91%, 92%, 93%, 94%, 95%, 96%, 97%, 98%, 99%, or 100% identical to the amino acid sequence of an one of SEQ NOS:1-2 or 11; and

(b) a multimerization domain capable of generating multimers comprising at least 60 copies of the isolated polypeptide.

In one embodiment, wherein the multimerization domain comprises an amino acid sequence at least 70%, 75%, 80%, 85%, 90%, 91%, 92%, 93%, 94%, 95%, 96%, 97%, 98%, 99%, or 100% identical to the amino acid sequence of SEQ IO NO: 3-4. In another embodiment:, the polypeptide further comprises an amino acid linker between the RBD and the multimerization domain.

In a further embodiment the polypeptide comprises amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 5-6, or 24, wherein n is 3-7, 3-6, 3-5, 3-4, 4-7, 4-6, 4-5, 5-7, 5-6, 3, 4, 5, 6, or 7. In one embodiment, the polypeptide comprises an aimno acid sequence at least 70%, 75%, 80%, 85%, 90%, 91%, 92%, 93%, 94%, 95%, 96%, 97%, 98%, 99%, or 100% identical to the amino acid sequence of any one of SEQ ID NOS:7-10 and 25-32.

In another embodiment, the disclosure provides multimers comprising 60 or more copies of a receptor binding domain (RBD) comprising an amino acid sequence at least 70%, 75%, 80%, 85%, 90%, 91%, 92%, 93%, 94%, 95%, 96%, 97%, 98%, 99%, or 100% identical to the amino acid sequence of any one of SEQ ID NOS:1-2 or 11. In one embodiment, the multimer comprises 60 or more copies of 1 or more polypeptides of the disclosure. another embodiment, the disclosure provides scaffolds, comprising 60 or more isolated polypeptides of any embodiment or combination of embodiments disclosed herein, on a surface of the scaffold, wherein the isolated polypeptides are all identical polypeptides, or wherein the isolated polypeptides include different polypeptides.

In other aspects, the disclosure provides nucleic acids encoding the isolated polypeptide of any embodiment or combination of embodiments disclosed herein, recombinant expression is comprisMg nucleic acids of die disclosure operatively linked to a suitable control sequence, and recombinant host cell comprising the polypeptide, the multimer, the scaffold, the nucleic acid, and/or the recombinant expression vector of any embodiment or combination of embodiments disclosed herein.

In one embodiment of the nucleic acids of the disclosure, the nucleic acid comprises mRNA. In another embodiment, the mRNA comprises a 5′ cap. In a further embodiment, the mRNA comprises a poly(A) tail of between 50 and 120 contiguous adenosine residues. In a still futrther embodiment, the mRNA comprises a 5′ untranslated region comprising the nucleic acid sequence of SEQ ID NO:12 or 13.In one embodiment, the mRNA comprises a 3′ untranslated region comprising or two copies of a beta globin mNRA 3′-UTR including but not limited to the nucleic acid sequence of SEC) ID NO:18. In another embodiment, the mRNA encodes a signal sequence, optionally whereinthe signal sequence is at the N-terminus of the encoded polypeptide, and option wherein the signal sequence comprises the amino acid sequence of SEQ ID NO 22 or 23.

In another embodiment, the disclosure provides composition comprising

(a) a plurality of polypeptides, multimers scatffolds of any one of claims 1-25, wherein the plurality of polypeptides, multimers or scaffolds include two or more different polypeptides of any embodiment or combination of embodiments disclosed herein; and/or

(b) a plurality of nucleic acids according to any embodiment or combination of embodiments disclosed herein, wherein the plurality of nucleic acids encode two or more different polypeptides of any embodiment or combination of embodiments disclosed

In one embodiment, the disclosure proyides pharmaceutical compositions, comprising

(a) the polypeptide, the multimer, the scaffold, the nucleic acid.,the recombinant expression vector, the cell, and/or the composition of any embodiment or combination of embodiments disclosed herein; and

(b) a pharmaceutically acceptable carrier.

In other aspects, the disclosure provides methods for treating or limiting development of a SARS coronavirus infection, comprising administering to a subject infected with or at risk of a SARS coronavirus an amount effective to treat or limit development of the infection of the polypeptide, the multimer, the scaffold, the nucleic acid, the recombinant expression vector, the cell, the composition, and/or the pharmaceutical composition of any embodiment or combination of embodiments disclosed herein.

In another aspect, the disclosure provides methods for generating an immune response in a subject, comprising administering to the subject an amount effective to generate an immune response of the polypeptide, the multimer, the scaffold, the nucleic acid, the recombinant expression vector, the cell, the composition, and/or the pharmaceutical composition of any embodiment or combination of embodiments disclosed herein. In one embodiment, the method comprises administering to the subject an amount effective of the pharmaceutical composition by subcutaneous, mtradermal or intramuscular injection. In another embodiment, the method comprises administering to the subject an effective amount Of the pharmaceutical composition with a needle-free injection system.

In a further aspect, the disclosure provides methods for monitoring a SARS coronavirus-induced disease in a subject andior monitoring response of the subject to immunization by a SARS coronavirus vaccine; comprising contacting the polypeptide, the multimer, the scaffold, the nucleic acid, the recombinant expression vector, the cell, the composition, andior the phatmaceutical composition of any embodiment or combination of embodiments disclosed herein with a bodily fluid from the subject and detecting SARS coronavirus-binding antibodies in the bodily fluid of the subject.

In one aspect, the disclosure provides methods fbr detecting SARS coronavirus binding antibodies, comprising

(a) contacting the polypeptide, the niultimer, the scaffold, and/or the pharmaceutical composition of any embodiment or combination of embodiments disclosed herein with a composition comprising a candidate SARS commivirus binding antibody under conditions duitable for binding of SARS coronavirus antibodies to the polypeptide, the multimer, the scaffold, and/or the pharmaceutical composition; and

(b) detecting SARS eoronaviilis antibody complexes with the polypeptide the multimer, the scaffold, and/or the pharmaceutical composition.

In another aspect, the disclosure provides methods for producing SARS coronavirus antibodies, comprising

(a) administering to a subject an amount effective to generate an antibody response of the polypeptide, the multimer, the scaffold, the nucleic acid, the recombinant expression vector, the cell, the composition, and/or the pharmaceutical composition of any embodiment Or combination of embodiments disclosed herein; and

(b) isolating antibodies produced by the subject.

### DESCRIPTION OF THE FIGURES

FIG. 1. RBD/ACE2 binding inhibition ELISA with VX3025r vaccine.

FIG. 2. SARS-CoV-2 neutralization test with VX3025r vaccine.

FIG. 3. RBD/ACE2 binding inhibition ELISA with bivalent N501Y wild type/alpha variant VX3025rB1 vaccine.

FIG. 4. Example of a multiplex mRNA-based composition according to the disclosure, with the multiplexing carried out by cells during translation of the mRNA.

### DETAILED DESCRIPTION

All references cited are herein incorporated by reference in their entirety. As used herein, the singular forms “a” “an” and the include plural referents unless the context dearly dictates otherwise.

All embodiments of any aspect of the disclosure can be used in combination, unless the context clearly dictates otherwise.

Unless the context:clearly requires othersvise, throughout the description and the claims, the words “comprise”, “comprising”, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein”, “above,” rand “below” and words of similar import, when used in this application, shall refer to this application as a whole and not to any particular portions of the application.

As used throughout the present application, the terms “protein” or “polypeptide” are used in their broadest sense to refer to a sequeree of subunit amino acids. The proteins or polypeptides of the disclosure may comprise L-amino acids, D-amino acids (which are resistant to L-amino acid-specific proteases in vivo), or a combination of D- and L-amino acids. The proteins or polypeptides described herein May be chemically synthesized or recombinantly expressed.

The description of embodiments of the disclosure is not intended to be exhaustive or to limit the disclosure to the precise form disclosed. While the, specific embodiments of, and examples for, the disclosure are described herein for illustrative purposes, various equivalent modifications are possible within the scope of the disclosure, as those skilled in the relevant art will recognize.

As used throughout the present application: the term “SARS coronavirus” is used in its broadest sense to designate any highly pathogenic coronavirus phylogenetically related to SARS-CoV or SARS-CoV-2.

As used herein, the amino acid residues are abbreviated as follows; alanine (Ala; A), asparagine (Asn; N), aspartic acid (Asp; D), arginine (Arg; R), cysteine (Cys; C), glutamic acid (Glu; E), glutamine (Gin; Q), glycine (Gly; G), histidine (His; H), isoleucine (Ile; I) leucine (Leu; L), lysine (Lys; K), methionine (Met; M ), phenylalamine (Phe; F) proline (Pro; P), serine (Ser; S), threonine (Thr; T), tryptophan (Trp; W), tyrosine (Tyr; Y), and valine (Val; V).

Parentheses represent variable positions in the polypeptide, with the recited amino acid residues as alternatives in these positions.

An abbreviated amino acid residue preceded or followed by a number indicates the position of the amino acid in a sequence of residues.

### Mutations of SARS-CoV-2 RBD

The following is a list of SARS-CoV-2 RBD mutations observed in humans in more than ten occurrences, or in single patients with prolonged infections as of Jul. 30, 2021:

R346K (Residue 16 in SEQ ID NO:1)

V367F (Residue 37 in SEQ ID NO:1)

R403K (Residue 73 in SEQ ID NO:1)

T415A (Residue 85 in SEQ ID NO:1)

K417N (Residue 87 in SEQ ID NO:1)

K417R (Residue 87 in SEQ ID NO:1)

K417T (Residue 87 in SEQ ID NO:1)

N439K (Residue 109 in SEQ ID NO:1)

V445A (Residue 115 in SEQ ID NO:1)

V445I (Residue 115 in SEQ ID NO:1)

G446S (Residue 116 in SEQ ID NO:1)

G446V (Residue 116 in SEQ ID NO:1)

Y449H (Residue 119 in SEQ ID NO:1)

Y449S (Residue 119 in SEQ ID NO:1)

L452Q (Residue 122 in SEQ ID NO:1)

L452R (Residue 122 in SEQ ID NO:1)

Y453F (Residue 123 in SEQ ID NO:1)

L455F (Residue 125 in SEQ ID NO:1)

F456L (Residue 126 in SEQ ID NO:1)

K458N (Residue 128 in SEQ ID NO:1)

T470N (Residue 140 in SEQ ID NO:1)

A475S (Residue 145 in SEQ ID NO:1)

A475V (Residue 145 in SEQ ID NO:1)

G476A (Residue 146 in SEQ ID NO:1)

G476S (Residue 145 in SEQ ID NO:1)

S477G (Residue 147 in SEQ ID NO:1)

S477I (Residue 147 in SEQ ID NO:1)

S477N (Residue 147 in SEQ ID NO:1)

S477R (Residue 147 in SEQ ID NO:1)

T478A (Residue 148 in SEQ ID NO:1)

T478I (Residue 148 in SEQ ID NO:1)

T478K (Residue 148 in SEQ ID NO:1)

T478R (Residue 148 in SEQ ID NO:1)

V483A (Residue 153 in SEQ ID NO:1)

E484A (Residue 154 in SEQ ID NO;1)

E484D (Residue 154 in SEQ ID NO:1)

E484K (Residue 154 in SEQ ID NO:1)

E484L (Residue 154 in SEQ ID NO:1)

E484Q (Residue 154 in SEQ ID NO:1)

G485K (Residue 155 in SEQ ID NO:1)

G485R (Residue 155 in SEQ ID NO:1)

F486I (Residue 156 in SEQ ID NO:1)

F490I (Residue 160 in SEQ ID NO:1)

F490S (Residue 160 in SEQ ID NO:1)

Q493K (Residue 163 in SEQ ID NO:1)

Q493L (Residue 163 in SEQ ID NO:1)

Q493R (Residue 163 in SEQ ID NO:1)

S494L (Residue 164 in SEQ ID NO:1)

S494P (Residue 164 in SEQ ID NO:1)

G496S (Residue 166 in SEQ ID NO:1)

N501T (Residue 171 in SEQ ID NO:1)

N501Y (Residue 171 in SEQ ID NO:1)

V503F (Residue 173 in SEQ ID NO:1)

V503I (Residue 173 in SEQ ID NO:1)

G504D (Residue 174 in SEQ ID NO:1)

Y505H (Residue 175in SEQ ID NO:1)

Y505W (Residue 175 in SEQ ID NO:1)

The above mutations are included in SEQ ID NO:1 of SARS-CoV-2 RBD variants:

(SEQ ID NO: 1)
NITNLCPFGEVFNAT(R/K)FASVYAWNRKRISNCVADYS(V/F)LYNSASFSTFKCYGVSP
TKLNDLCFTNVYADSFVI(R/K)GDEVRQIAPGQ(T/A)G(K/N/R/T)IADYNYKLPDDFT
GCVIAWNS(N/K)NLDSK(V/A/I)(G/S/V)GN(Y/H/S)NY(L/Q/R)(Y/F)R(L/F)
(F/L)R(K/N)SNLKPFERDIS(T/N)EIYQ(A/S/V)(G/A/S)(S/G/I/N/R)(T/A/I/
K/R)PCNG(V/A)(E/A/D/K/L/Q)(G/K/R)(F/I)NCY(F/L/S)PL(Q/K/L/R)(S/
L/P)Y(G/S)FQPT(N/T/Y)G(V/F/I)(G/D)(Y/H/W)QPYRVVVLSFELLHAPATV
(SARS-COV-2 RBD Variants)
SEQ ID NO: 2 is the reference sequence of SARS-COV-2 RBD.
(SEQ ID NO: 2)
NITNLCPFGEVFNATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKVGGNYNYLYR
LLHAPATV (SARS-COV-2 RBD; residues 331-524 in the S1 subunit)

In one aspect, the disclosure provides isolated polypeptide comprising:

(a) a receptor binding domain (RBD) comprising an amino acid sequence at least 70%, 75%, 80%, 85%, 90%, 91%, 92%, 93%, 94% 95%, 97%, 98%, or 100% identical to the amino acid sequence of any one of SEQ ID NOS:1-2; and

(b) a multimerization domain capable of generating multimers comprising at least 61 copies of the: isolated polypeptide.

In one embodiment, the isolated polypeptide comprises an RBD comprising an amino acid sequence at least 70%, 75%, 80%, 85%, 90%, 91%, 92%, 93%, 94% 95%, 97%, 98%, or 100% identical to the amino acid sequence of SEQ ID NO: 11.

(SEQ ID NO: 11)
NITNLCPFGEVFNATRFASVYAWNRKRISNCVADYSVLYNSASFSTFKCYGVSPTKLNDLCF
TNVYADSFVI(R/K)GDEVRQIAPGQTG(K/N)IADYNYKLPDDFTGCVIAWNS(N/K)NLD
SK(V/I/A)(G/V/S)GNYNYL(Y/F)R(L/F)(F/L)RKSNLKPFERDISTEIYQ(A/V)
(G/S/A)(S/N/I/G/R)(T/I/A/K)PCNGV(E/Q/K/A/L/D)(G/R/K)FNCY(F/S/L)
PL(Q/L)(S/P/L)YGFQPT(N/Y)GV(G/D)(Y/W)QPYRVVVLSFELLHAPATV
(SARS-COV-2 RBD Variants embodiment 2)

This embodiment is based on the following list of RBD mutations:

R403K (Residue 73 in SEQ ID NO:1)

K417N (Residue 87 in SEQ ID NO:1)

N439K (Residue 109 in SEQ ID NO:1)

V445A (Residue 115 in SEQ ID NO:1)

V445I (Residue 115 in SEQ ID NO:1)

G446S (Residue 116 in SEQ ID NO:1)

G446V (Residue 116 in SEQ ID NO:1)

Y453F (Residue 123 in SEQ ID NO:1)

L455F (Residue 125 in SEQ ID NO:1)

F456L (Residue 126 in SEQ ID NO:1)

A475V (Residue 145 in SEQ ID NO:1)

G476N (Residue 146 in SEQ ID NO:1)

G476S (Residue 146 in SEQ ID NO:1)

S477G (Residue 147 in SEQ ID NO:1)

S477I (Residue 147 in SEQ ID NO:1)

S477N (Residue 147 in SEQ ID NO:1)

S477R (Residue 147 in SEQ ID NO:1)

T478A (Residue 148 in SEQ ID NO:1)

T478I (Residue 148 in SEQ ID NO:1)

T478K (Residue 148 in SEQ ID NO:1)

E484A (Residue 154 in SEQ ID NO:1)

E484D (Residue 154 in SEQ ID NO:1)

E484I (Residue 154 in SEQ ID NO:1)

E484I (Residue 154 in SEQ ID NO:1)

E484Q (Residue 154 in SEQ ID NO:1)

G485K (Residue 155 in SEQ ID NO:1)

G485R (Residue 155 in SEQ ID NO:1)

F490I (Residue 100 in SEQ ID NO:1)

F490S (Residue 160 in SEQ ID NO:1)

Q493L (Residue 163 in SEQ ID NO:1)

S494L (Residue 164 in SEQ ID NO:1)

S494P (Residue 164 in SEQ ID NO:1)

N501Y (Residue 171 in SEQ ID NO:1)

G504D (Residue 174 in SEQ ID NO:1)

Y505W (Residue 175 in SEQ ID NO:1)

In another embodiment, the isolated polypeptide comprises an RBD comprising an

amino add sequence at least 70%, 75%, 80%, 85%, 90%, 91%, 92%, 93%, 94%, 95%, 96%. 97%. 98%, 99%, or 100% identical to the amino acid sequence oI SEQ ID NO:2, wherein the RBD comprises at least at 1, 2, 3, 4, 5, 6, 7, 8, 9, 10, 11, 12. 13, 14, 15, 16, 17, 18, 19, 20, 21, 22, 23, 24, 25, 26, 27, 28, 29, 30, or 31 of the following mutations at different residues relative to SEQ ID NO:2:

R346K (Residue 16 in SEQ ID NO:2)

V367F (Residue 37 in SEQ ID NO:2)

R403K (Residue 73 in SEQ ID NO:2)

T415A (Residue 85 in SEQ ID NO:2)

K417N (Residue 87 in SEQ ID NO:2)

K417R (Residue 87 in SEQ ID NO:2)

K417T (Residue 87 in SEQ ID NO:2)

N439K (Residue 109 in SEQ ID NO:2)

V445A (Residue 115 in SEQ ID NO:2)

V445I (Residue 115 in SEQ ID NO:2)

G446S (Residue 116 in SEQ ID NO:2)

G446V (Residue 116 in SEQ ID NO:2)

Y449H (Residue 119 in SEQ ID NO:2)

Y449S (Residue 119 in SEQ ID NO:2)

L452Q (Residue 122 in SEQ ID NO:2)

L452R (Residue 122 in SEQ ID NO:2)

Y453F (Residue 123 in SEQ ID NO:2)

L455F (Residue 125 in SEQ ID NO:2)

F456L (Residue 126 in SEQ ID NO:2)

K458N (Residue 128 in SEQ ID NO:2)

T470N (Residue 140 in SEQ ID NO:2)

A475S (Residue 145 in SEQ ID NO:2)

A475V (Residue 145 in SEQ ID NO:2)

G476A (Residue 146 in SEQ ID NO:2)

G476S (Residue 146 in SEQ ID NO:2)

S477G (Residue 147 in SEQ ID NO:2)

S477I (Residue 147 in SEQ ID NO:2)

S477N (Residue 147 in SEQ ID NO:2)

S477R (Residue 147 in SEQ ID NO:2)

T478A (Residue 148 in SEQ ID NO:2)

T478I (Residue 148 in SEQ ID NO:2)

T478K (Residue 148 in SEQ ID NO:2)

T478R (Residue 148 in SEQ ID NO:2)

V483A (Residue 153 in SEQ ID NO:2)

E484A (Residue 154 in SEQ ID NO:2)

E484D (Residue 154 in SEQ ID NO:2)

E484K (Residue 154 in SEQ ID NO:2)

E484I (Residue 154 in SEQ ID NO:2)

E484Q (Residue 154 in SEQ ID NO:2)

G485K (Residue 155 in SEQ ID NO:2)

G485R (Residue 155 in SEQ ID NO:2)

F486I (Residue 156 in SEQ ID NO:2)

F490I (Residue 160 in SEQ ID NO:2)

F490S (Residue 160 in SEQ ID NO:2)

Q493K (Residue 163 in SEQ ID NO:2)

Q493I (Residue 163 in SEQ ID NO:2)

Q493R (Residue 163 in SEQ ID NO:2)

S494L (Residue 164 in SEQ ID NO:2)

S494P (Residue 164 in SEQ ID NO:2)

G496S (Residue 166 in SEQ ID NO:2)

N501T (Residue 171 in SEQ ID NO:2)

N501T (Residue 171 in SEQ ID NO:2)

V503F (Residue 173 in SEQ ID NO:2)

V503I (Residue 173 in SEQ ID NO:2)

G504D (Residue 174 in SEQ ID NO:2)

Y505H (Residue 175 in SEQ ID NO:2)

Y505W (Residue 175 in SEQ ID NO:2)

The above list includes mutations at 30 residues in the RBD of SEQ ID. NO:2, with some residues having multiple mutations listed. Those of skill in the art will understand that a single polypeptide will have only One mutation at a given residue (i.e., the polypeptide comprises at least at 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the following mutations at different residues relative to SEQ ID NO:2).

In all embodiments disclosed herein, polypeptides may comprise additional mutations not listed relative to the reference. RBD amino acid sequence, so long as it meets the percent identity requirement.

In another embodiment, the isolated polypeptide comprises an RBD comprising an ammo acid sequence at least 85%, 90%, 91%, 92% , 93%, 94%, 97%, 98%, 99%, or 100% identical to the amino acid sequence of SEQ ID NO:2, wherein the

RBD Comprises at least at 1, 2, 3, 4, 5, 6, 7, 8, 9, 10, 11, 12, 13, 14, 15, 16, 17, 18, 19, 20, 21, 22, 23, 24, 25, 26, 27, 28, or 29 of the following mutations at diff rent residues relative to SEQ ID NO:2:

R346K (Residue 16 in SEQ ID NO:2)

V367F (Residue 37 in SEQ ID NO:2)

R403K (Residue 73 in SEQ ID NO:2)

T415A (Residue 85 in SEQ ID NO:2)

K417N (Residue 87 in SEQ ID NO:2)

K417R (Residue 87 in SEQ ID NO:2)

K417T (Residue 87 in SEQ ID NO:2)

N439K (Residue 109 in SEQ ID NO:2)

V445A (Residue 115 in SEQ ID NO:2)

G446S (Residue 116 in SEQ ID NO:2)

G446V (Residue 116 in SEQ ID NO:2)

Y449H (Residue 119 in SEQ ID NO:2)

Y449S (Residue 119 in SEQ ID NO:2)

L452Q (Residue 122 in SEQ ID NO:2)

L452R (Residue 122 in SEQ ID NO:2)

Y453F (Residue 123 in SEQ ID NO:2)

L455F (Residue 125 in SEQ ID NO:2)

F456L (Residue 126 in SEQ ID NO:2)

K458N (Residue 128 in SEQ ID NO:2)

T470N (Residue 140 in SEQ ID NO:2)

A475S (Residue 145 in SEQ ID NO:2)

A475V (Residue 145 in SEQ ID NO:2)

G476S (Residue 146 in SEQ ID NO:2)

S477G (Residue 147 in SEQ ID NO:2)

S477I (Residue 147 in SEQ ID NO:2)

S477N (Residue 147 in SEQ ID NO:2)

S477R (Residue 147 in SEQ ID NO:2)

T478I (Residue 148 in SEQ ID NO:2)

T478I (Residue 148 in SEQ ID NO:2)

T478R (Residue 148 in SEQ ID NO:2)

V483A (Residue 153 in SEQ ID NO:2)

E484K (Residue 154 in SEQ ID NO:2)

E484Q (Residue 154 in SEQ ID NO:2)

F486I (Residue 156 in SEQ ID NO:2)

F490S (Residue 160 in SEQ ID NO:2)

Q493K (Residue 163 in SEQ ID NO:2)

Q493R (Residue 163 in SEQ ID NO:2)

S494L (Residue 164 in SEQ ID NO:2)

S494P (Residue 164 in SEQ ID NO:2)

G496S (Residue 166 in SEQ ID NO:2)

N501T (Residue 171 in SEQ ID NO:2)

N501Y (Residue 171 in SEQ ID NO:2)

V503F (Residue 173 in SEQ ID NO:2)

V503I (Residue 173 in SEQ ID NO:2)

Y503H (Residue 175 in SEQ ID NO:2)

In a further embodiment, the isolated polypeptide comprises an RBD comprising an amino acid sequence at least 70%, 75%, 80%, 85%, 91%, 92%, 93%, 94%, 95%, 96%, 97%, 98%, 99%, or 100% identical to the amino acid sequence of SEQ ID NO:2, wherein the RBD comprises at least at 1, 2, 3, 4, 5, 6, 7, or 8 of the following mutations at different residues relative to SEQ ID NO:2:

R346K (Residue 16 in SEQ ID NO:2)

V367F (Residue 37 in SEQ ID NO:2)

K417N (Residue 87 in SEQ ID NO:2)

K417T (Residue 87 in SEQ ID NO:2)

L452Q (Residue 122 in SEQ ID NO:2)

L452R (Residue 122 in SEQ ID NO:2)

T478K (Residue 148 in SEQ ID NO:2)

T478R (Residue 148 in SEQ ID NO:2)

E484K (Residue 154 in SEQ ID NO:2)

E484Q (Residue 154 in SEQ ID NO:2)

F490S (Residue 160 in SEQ ID NO:2)

N501Y (Residue 171 in SEQ ID NO:2)

Since December 2020 the World Health Organization has classified SARS-CoV-2 variants as i) Variants of Concern (VOC), it they are associated with an increase of transmissibility or rulence, or a decrease of effectiveness of available vaccines or therapeutics; and ii) Variants of Interest (VOI) if they are identified to cause community transmission, or multiple cases or clusters, or detected in multiple countries. The following lists the RBD mutations of VOCs and VOIs as of Aug. 13, 2021:

| Variants of Concern | |
|---|---|
| Alpha (B.1.1.7) | N501Y |
| Beta (B.1.351) | K417N, E484K, N501Y |
| Gamma (P.1) | K417T, E484K, N501Y |
| Delta (B.1.617.2) | L452R, T478K |

| Variants of Interest | |
|---|---|
| Epsilon (B.1.429) | L452R |
| Zeta (P.2) | E484K |
| Eta (B.1.525) | E484K |
| Theta (P.3) | E484K, N501Y |
| Iota (B.1.526) | E484K |
| Kappa (B.1.617.1) | L452R, E484Q |
| Lambda (B.1.1.1) | L452Q, F490S |

It can be observed that there is a convergence of RBD mutations of VOCs and VOIs. For example E48 4K is observed in 6 variants, N501Y in 4 variants, L452R in 3 variants, and only 6 other different RBD mutations are observed in all VOCs and VOIs. Also 10 VOCs and VCRs carry imitations at only 5 residues.

The polypeptides of the disclosure comprise a multimerization domain. For example, the polypeptides can be engineered via genetic fusion to create 60-mer multimers. These constructs may be expressed, for example, in Chinese hamster ovary (CHO) cells and purified using standard nickel and size exclusion methods, B size exclusion chromatography with multi-angle light scattering (SEC-MALS), each construct is shown to have the correct molecular weight according to its intended multimeric state. The antigenic profiles of the constructs are tested and the results show binding to neutralizing antibodies.

The polypeptide is capable of multimerization and thus presenting, multiple copies of the RBD to enhance the immune response generated when the polypeptide is administered to a subject. Any multimerization can used that is capable of generating multimers comprising at least 60 copies of the isolated polypeptide, and as deemed suitable fix an intended use. In one embodiment, the multimerization domain comprises an amino acid sequence at least at least 70%, 75%, 80%, 85%, 90%, 91%, 92%, 93%, 94%, 95%, 96%, 97%, 98%, 99%, or 100% identical to the amino acid sequence of SEQ ID NO: 3 or 4.

(SEQ ID NO: 3)
MQIY(E/C)GK(L/C)(T/G)AEGLRFGIVASR(F/A)NHALVDELVEGAIDAIV(R/C)
(H/F/M)GGREEDITLV(R/C)V(P/C)GSWEIP(V/C)AAGELARKEDIDAVIAIGVL(I/C)
RGA(T/C)(P/G)(H/S)FDYIASEVSKGLADLS(L/C)ELRKPITFGVITA(D/C)TLEQA
IE(R/A)AGT(K/C)HGNKGWEAAL(S/C)AIEMANLEKSLR (Lumazine
synthase (LS) variants)
(SEQ ID NO: 4)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLR (Lumazine synthase (LS))

In this embodiment, the multimerization platform comprises lunrazine synthase. The imiltimerization domains of SEQ ID NOS: 3 and 4 can be used to generate multimers comprising 60 copies of the isolated polypeptides of the disclosure.

In one embodiment where the linker comprises SEQ ID NO:3, at least 1, 2, 3, 4, 5, 6, 7, 8, 9, 10, 11, 12, 13, 14, 15, 16, 17 or all 18 of the residues bounded by parentheses is the first listed residue.

In another embodiment the polypeptides of the disclosure may further comprise an amino acid linker between the RBD and the multimerization domain. Any amino acid linker may be used as suitable for an intended purpost., one embodiment, the linker is a Gly-Ser rich linker (i.e.: 50%, 60%, 70%, 80%, 90%, 95%, or 100% made up of Gly or Ser residues), The combination of flexible and hydrophilic residues in these linkers limits the formation of secondary structures and reduces the likelihood that the linkers will interfere with the folding and function of the protein domains. In one specific embodiment, the linker comprises or consists of (GGS)ₙGGG, wherein n is 3-7, 3-6, 3-5, 3-4, 4-7, 4-6, 4-5, 5-7, 5-6, 3, 4, 5, 6, or 7.

The multimerization may be N-tenninal or C-terminal to the RBD. In one specific embodiment, the RBD is carboxy-tenninal to the multimerization domain.

In other embodiments, the polypeptide comprises the amino acid sequence selected from the group consisting of SEQ ID NOS: 5-6 or 24, wherein n is 3-7, 3-6, 3-5, 3-4, 4-7, 4-6, 4-5, 5-7, 5-6, 3, 4, 5, 6, or 7.

(SEQ ID NO: 5)
MQIY(E/C)GK(L/C)(T/G)AEGLRFGIVASR(F/A)NHALVDRLVEGAIDAIV(R/C)
(H/F/M)GGREEDITLV(R/C)V(P/C)GSWEIP(V/C)AAGELARKEDIDAVIAIGVL(I/C)
RGA(T/C)(P/G)(H/S)FDYIASEVSKGLADLS(L/C)ELRKPITFGVITA(D/C)TLEQA
IE(R/A)AGT(K/C)HGNKGWEAAL(S/C)AIEMANLFKSLR(GGS)ₙGGGNITNLCPFGEVF
NATRFASVYAWNRKRISNCVADYSVLYNSASFSTFKCYGVSPTKLNDLCFTNVYADSFVI
(R/K)GDEVRQIAPGQTG(K/N)IADYNYKLPDDFTGCVIAWNS(N/K)NLDSK(V/I/A)(G/
V/S)GNYNYL(Y/F)R(L/F)(F/L)RKSNLKPFERDISTEIYQ(A/V)(G/S/A)(S/N/I/
G/R)(T/I/A/K)PCNGV(E/Q/K/A/L/D)(G/R/K)FNCY(F/S/L)PL(Q/L)(S/P/L)
YGFQPT(N/Y)GV(G/D)(Y/W)QPYRVVVLSFELLHAPATV
(SEQ ID NO: 24)
MQIY(E/C)GK(L/C)(T/G)AEGLRFGIVASR(F/A)NHALVDELVEGAIDAIV(R/C)
(H/F/M)GGREEDITLV(R/C)V(P/C)GSWEIP(V/C)AAGELARKEDIDAVIAIGVL(I/C)
RGA(T/C)(P/G)(H/S)FDYIASEVSKGLADLS(L/C)ELRKPITFGVITA(D/C)TLEQA
IE(R/A)AGT(K/C)HGNKGWEAAL(S/C)AIEMANLFKSLR(GGS)ₙGGGNITNLCPFGEVF
NAT(R/K)FASVYAWNRKRISNCVADYS(V/F)LYNSASFSTFKCYGVSPTKLNDLCFTNVY
ADSFVI(R/K)GDEVRQIAPGQ(T/A)G(K/N/R/T)IADYNYKLPDDFTGCVIAWNS(N/K)
NLDSK(V/A/I)(G/S/V)GN(Y/H/S)NY(L/Q/R)(Y/F)R(L/F)(F/L)R(K/N)SN
LKPFERDIS(T/N)EIYQ(A/S/V)(G/A/S)(S/G/I/N/R)(T/A/I/K/R)PONG(V/A)
(E/A/D/K/L/Q)(G/K/R)(F/I)NCY(F/L/S)PL(Q/K/L/R)(S/L/P)Y(G/S)FQ
PT(N/T/Y)G(V/F/I)(G/D)(Y/H/W)QPYRVVVLSFELLHAPATV
(SEQ ID NO: 6)
MQIY(E/C)GK(L/C)(T/G)AEGLRFGIVASR(F/A)NHALVDRLVEGAIDAIV(R/C)
(H/F/M)GGREEDITLV(R/C)V(P/C)GSWEIP(V/C)AAGELARKEDIDAVIAIGVL(I/C)
RGA(T/C)(P/G)(H/S)FDYIASEVSKGLADLS(L/C)ELRKPITFGVITA(D/C)TLEQA
IE(R/A)AGT(K/C)HGNKGWEAAL(S/C)AIEMANLFKSLR(GGS)nGGGNITNLCPFGEVF
NATRFASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSNLKPFER
DISTEIYQAGSTPCNGVEGFNCYFPLQSYGFQPTNGVGYQPYRVVVLSFELLHAPATV

In specific embodiments, the polypeptide comprises the amino acid sequence of any one of SEQ ID NOS:7-10 and 25-32.

(SEQ ID NO: 7)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGNITNLCPFGEVFNATRF
ASVYAWNRKRISNCVADYSVLYNSASFSTFKCYGVSPTKLNDLCFTNVYADSFVI(R/K)GD
EVRQIAPGQTG(K/N)IADYNYKLPDDFTGCVIAWNS(N/K)NLDSK(V/I/A)(G/V/S)G
NYNYL(Y/F)R(L/F)(F/L)RKSNLKPFERDISTEIYQ(A/V)(G/S/A)(S/N/I/G/R)
(T/I/A/K)PCNGV(E/Q/K/A/L/D)(G/R/K)FNCY(F/S/L)PL(Q/L)(S/P/L)YGF
QPT(N/Y)GV(G/D)(Y/W)QPYRVVVLSFELLHAPATV
(SEQ ID NO: 25)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGNITNLCPFGEVFNAT
(R/K)FASVYAWNRKRISNOVADYS(V/F)LYNSASFSTFKCYGVSPTKLNDLCFTNVYADSFV
I(R/K)GDEVRQIAPGQ(T/A)G(K/N/R/T)IADYNYKLPDDFTGCVIAWNS(N/K)NLDS
K(V/A/I)(G/S/V)GN(Y/H/S)NY(L/Q/R)(Y/F)R(L/F)(F/L)R(K/N)SNLKPFE
RDIS(T/N)EIYQ(A/S/V)(G/A/S)(S/G/I/N/R)(T/A/I/K/R)PCNG(V/A)(E/A/
D/K/L/Q)(G/K/R)(F/I)NCY(F/L/S)PL(Q/K/L/R)(S/L/P)Y(G/S)FQPT(N/T/Y)
G(V/F/I)(G/D)(Y/H/W)QPYRVVVLSFELLHAPATV
(SEQ ID NO: 8)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGRFPNITNLCPFGEVFNA
TRFASVYAWNRKRISNCVADYSVLYNSASFSTFKCYGVSPTKLNDLCFTNVYADSFVI(R/K)
GDEVRQIAPGQTG(K/N)IADYNYKLPDDFTGCVIAWNS(N/K)NLDSK(V/I/A)(G/V/S)
GNYNYL(Y/F)R(L/F)(F/L)RKSNLKPFERDISTEIYQ(A/V)(G/S/A)(S/N/I/G/
R)(T/I/A/K)PCNGV(E/Q/K/A/L/D)(G/R/K)FNCY(F/S/L)PL(Q/L)(S/P/L)
YGFQPT(N/Y)GV(G/D)(Y/W)QPYRVVVLSFELLHAPATVCGPKKST
(SEQ ID NO: 26)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGRFPNITNLCPFGEVFNA
T(R/K)FASVYAWNRKRISNCVADYS(V/F)LYNSASFSTFKCYGVSPTKLNDLCFTNVYAD
SFVI(R/K)GDEVRQIAPGQ(T/A)G(K/N/R/T)IADYNYKLPDDFTGCVIAWNS(N/K)N
LDSK(V/A/I)(G/S/V)GN(Y/H/S)NY(L/Q/R)(Y/F)R(L/F)(F/L)R(K/N)SNLK
PFERDIS(T/N)EIYQ(A/S/V)(G/A/S)(S/G/I/N/R)(T/A/I/K/R)PCNG(V/A)
(E/A/D/K/L/Q)(G/K/R)(F/I)NCY(F/L/S)PL(Q/K/L/R)(S/L/P)Y(G/S)FQPT
(N/T/Y)G(V/F/I)(C/D)(Y/H/W)QFYRVVVLSFELLHAPATVCGPKKST
(SEQ ID NO: 9)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEQQGTCHGNKGWEAALCAIEMANLFKSLRGGSGGSGGSGGSGGGNITNLCPFGEVFNATRF
ASVYAWNRKRISNCVADYSVLYNSASFSTFKCYGVSPTKLNDLCFTNVYADSFVIRGDEVRQ
IAPGQTGKIADYNYKLPDDFTGCVIAWNSNNLDSKVGGNYNYLYRLFRKSNLKPFERDISTE
IYQAGSTPCNGVEGFNCYFPLQSYGFQPINGVGYQPYRVVVLSFELLHAPATV
(SEQ ID NO: 27)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGNITNLCPFGEVFNAT
(R/K)FASVYAWNRKRISNCVADYS(V/F)LYNSASFSTFKCYGVSPTKLNDLCFTNVYADSFV
IRGDEVRQIAPGQTG(K/N/T)IADYNYKLPDDFTGCVIAWNSNNLDSKVGGNYNY(L/Q/R)
YRLFRKSNLKPFERDISTEIYQAGS(T/K/R)PCNGV(E/K/Q)GFNCY(F/S)PLQSYGF
QPT(N/Y)GVGYQPYRVVVLSFELLHAPATV
(SEQ ID NO: 10)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGRFPNITNLCPFGEVFNA
TRFASVYAWNRKRISNCVADYSVLYNSASFSTFKCYGVSPTKLNDLCFTNVYADSFVIRGDE
VRQIAPGQTGKIADYNYKLPDDFTGCVIAWNSNNLDSKVGGNYNYLYRLFRKSNLKPFERDI
STEIYQAGSTPCNGVEGFNCYFPLQSYGFQPTNGVGYQPYRVVVLSFELLHAPATVCGPKKS
T
(SEQ ID NO: 28)
MQIYEGKLTAEGLRFGIVASRANHALVDRLVEGAIDAIVRHGGREEDITLVRVCGSWEIPVA
AGELARKEDIDAVIAIGVLCRGATPSEDYIASEVSKGLADLSLELRKPITFGVITADTLEQA
IEAAGTCHGNKGWEAALCAIEMANLFKSLRGGSGGSGGSGGSGGGRFPNITNLCPFGEVFNA
T(R/K)FASVYAWNRKRISNCVADYS(V/F)LYNSASFSTFKCYGVSPTKLNDLCFTNVYAD
SFVIRGDEVRQIAPGQTG(K/N/T)IADYNYKLPDDFTGCVIAWNSNNLDSKVGGNYNY(L/
Q/R)YRLFRKSNLKPFERDISTEIYQAGS(T/K/R)PCNGV(E/K/Q)GFNCY(F/S)PLQS
YGFQPT(N/Y)GVGYQPYRVVVLSFELLHAPATVCGPKKST
(SEQ ID NO: 29)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGRFPNITNLCPFGEVFNA
TRFASVYAWNRKRISNCVADYSVLYNSASFSTEKCYGVSPTKLNDLCFTNVYADSFVIRGDE
VRQIAPGQTGKIADYNYKLPDDFTGCVIAWNSNNLDSKVGGNYNYLYRLFRKSNLKPFERDI
STEIYQAGSTPCNGVEGENCYFPLQSYGFQPTYGVGYQPYRVVVLSFELLHAPATVCGPKKS
T
(SEQ ID NO: 30)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGRFPNITNLCPFGEVFNA
TRFASVYAWNRKRISNCVADYSVLYNSASESTFKCYGVSPTKLNDLCFTNVYADSFVIRGDE
VRQIAPGQTGNIADYNYKIPDDFTGCVIAWNSNNLDSKVGGNYNYLYRLFRKSNLKPFERDI
STEIYQAGSTPCNGVKGENCYFPLQSYGFQPTYGVGYQPYRVVVLSFELLHAPATVCGPKKS
T
(SEQ ID NO: 31)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGRFPNITNLCPFGEVENA
TRFASVYAWNRKRISNCVADYSVLYNSASFSTEKCYGVSPTKLNDLCFTNVYADSFVIRGDE
VRQIAPGQTGTIADYNYKLPDDETGCVIAWNSNNLDSKVGGNYNYLYRLFRKSNLKPFERDI
STEIYQAGSTPCNGVKGFNCYFPLQSYGFQPTYGVGYQPYRVVVLSFELLHAPATVCGPKKS
T
(SEQ ID NO: 32)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGRFPNITNLCPFGEVFNA
TRFASVYAWNRKRISNCVADYSVLYNSASFSTFKCYGVSPTKLNDLCFTNVYADSFVIRGDE
VRQTAPGQTGKIADYNYKLPDDETGCVIAWNSNNLDSKVGGNYNYRYRLFRKSNLKPFERDI
STEIYQAGSKPCNGVEGFNCYFPLQSYGFQPTNGVGYQPYRVVVLSFELLHAPATVCGPKKS
T

The polypeptides may include additional sequences/functional domains at the N- or C-termini as deemed appropriate for an intended use, including but not limited to detectable tags. domains to facilitate protein purification, etc. In one embodiment, the polypeptides may further comprise a signal sequence. Any suitable signal sequence may be used. In one embodiment, the signal sequence is encoded at the N-terminus of the polypeptide. In a non-limiting embodiment, the signal sequence may comprise the human interleukin-2 signal peptide MYRMQLLSCIALSLALVTNS (SEQ ID NO:23), which may optionally be present at the N-terminus of the polypeptide.

In another aspect, the disclosure provides multimers, comprising two or more copies of the isolated polypeptide of any embodiment or combination of embodiments disclosed herein. The multitmers may be formed in any suitable manner, including but not limited to by inclusion of multimerization domains in the primary amino acid sequence, or by linking the polypeptides to a scaffold. In one embodiment, the multimer comprises between 2 and 60 copies of the isolated polypeptide. In various embodiments, the multimer may comprise 2, 3, 4, 6 8, 60, or more copies of the polpeptide. In one embodiment, the disclosure provides scaffolds comprising two or more isolated polypeptides of any embodiment or combination of embodiments disclosed herein on a surface of the scaffold. Any suitable scaffolds may be used, whether polypeptide scaffolds, virus-like particles, beads, or other scaffold materials. The polypeptides may be linked to the scaffolds in any suitable MaitCr. In ane embodiment., the two or more isolated polypeptides are all identical polypeptides, lai another embodiment, the two or more isolated polypeptides include 2, 3, 4, 5 6, 7, 8, 9, 10, 11, 12, 13, 14, 15, 16, 17, 18, 19, 20, or more different polypeptides, permitting delivery of a multivalent composition to a subject in need thereof. As shown in the examples that follow, there is no loss of immunogenicity in multivalent compositions as compared to each monovalent component. Convergence of mutations in the RBD indicates that a small valency can cover a large number of variants, and thus these multivalent compositions provide a significant clinical benefit.

In one embodiment, the two or more isolated polypeptides in the multimers or scaffolds comprises 2, 3, 4, or more polypeptides comprising an amino acid sequence at least 70%, 75%, 80%, 85%, 90%, 91%, 92%, 93%, 94%, 95%, 96%, 97c/i, 98%, 99%, or 100% identical to the amino acid sequence of any one of SEQ ID NOS:7-10 and 25-32. In another embodiment, the two or more isolated polypeptides in the multimers or scaffolds comprises 2, 3, or all 4 polypeptides comprising an amino acid sequence at least 70%, 75%, 80%, 85%, 90%, 91%, 92%, 93%, 94%, 95%, 96%, 97%, 98%, 99%, or 100% identical to the amino acid sequence of SEQ ID NOS:29-32. In a still further embodiment, the two or more isolated polypeptides in the multimers or scaffolds comprises 2, 3, or all 4 polypeptides eon/prising the amino acid sequence of SEQ ID NOS:29-32.

In another aspect, the disclosure provides isolated nucleic acids encoding the isolated polypeptide of any embodiment or combination of embodiments disclosed herein. The isolated nucleic acid sequence may comprise RNA or DNA. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In another aspect, the present disclosure provides expression vectors comprising the nucleic acid of any aspect of the disclosure operatively linked to a suitable control sequence, “Expression vector” includes vectors that operatively link a nucleic acid coding region or gene to any control sequences capable of effecting expression of the gene product, “Control sequences” operably linked, to the nucleic acid sequences of the invention are nucleic acid sequences capable of effecting the expression of the nucleic acid molecules. The control sequences need not be contiguous with the nucleic acid sequences, so long as they function to direct the expression the.reof Thus, for exampl: intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors include but are not limited to, plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector (including but not limited to a retroviral vector or oncolytic virus), or any other suitable expression vector. In some embodiments, the expression vector can be administered in the methods of the disclosure to ex-press the polypeptides in vivo for therapeutic benefit.

In a further aspect, the present disclosure provides host cells that comprise the polypeptides, nucleic acids, expression vectors and/or nucleic acids disclosed herein, wherein the host cells can be either prokaryotic or eukaryotic. The cells can be transiently or stably engineered to incorporate. the expression vector of the invention, using techniques including but not limited to bacterial minsforrnations, calcium phosphate co-precipitation, electroporation, liposome mediated-, DEAE dextran mediated-, polyeationic mediated-, or viral mediated transfection. (See, for example, Molecular Cloning: A Laboratory Manual (Sambrook, et al., 1989 Cold Spring Harbor Laboratory Press); Culture of Animal Cells: A Manual of Basic Technique, 2^{nd} Ed. (R. I. Freslney, 1987, Liss, Inc. New York, NY)). A method of producing a polypeptide according to the invention is an additional part of the invention. The method comprises the steps of (a) culturing a host according to this aspect of the invention under conditions conducive to the expression srfthe polypeptide, and (b) optionally recovering the expressed polypeptide. The expressed polypeptide can be recovered from the cell free extract, but preferably they are recovered from the culture medium.

In one embodiment of the nucleic acids of the disclosure, the nucleic acid comprises mRNA. Messenger RNA (mRNA) offers a relatively safe and efficient alternative to the polypeptide therapeutics fitrid vaccines of the disclosure. After rRNA in vivo injection find uptake by professional antigen-presenting cells (APCs) in various tissues the RBD is expressed in APCs and displayed for the immune response.

Various modifications of mRNA may be used in order to counterthe degradation of a RBD mRNA therapeutic or vaccine disclosed herein.

In one embodiment, the mRNA comprises a 5′ cap. The 5′ cap is a specially altered nucleotide on the 5′ end of mRNA. This process, known as mRNA capping, is highly regulated and vital in the creation of stable and mature messenger RNA able to undergo translation during protein synthesis. In eukaaryotes the 5′ cap consists of a guanine nucleotide connected to mRNA via an unusual 5′ to 5′ triphosphate linkage. This guanosine is methylated directly after capping in rho by a methyltransferase. In multieellular eukaryotes further modifications exist, including the methylation of the first 2 ribose sugars of the 5′ end, of the mRNA. The 5′ cap is chemically similar to the 3′ end of an RNA molecule and this provides significant resistance to 5′exonucleases, Eukaryotic RNA undergoes a series of modifications in order to be exported from the nucleus and successfully translated into function proteins, many of which are dependent on mRNA capping, the first mRNA modification to take place. Various versions of 5′ caps can be added during or after the transcription reaction using various capping enzymes such as a vaccinia virus capping enzyme or by incorporating a synthetic cap or anti-reverse cap analogues.

In another embodiment, the mRNA further comprises a poly(A) tail of between 50 and 120 contiguous adenosine residues. Polyadenylation helps protect the mRNA 3′ end against degradation by exonucleases, the export of mature mRNA to the cytoplasmic environment, and also for mRNA translation.

In another embodiment, the mRNA comprises a 5′ untranslated region (including the start codon) comprising the sequence GGGAGACUGCCACCAUG (SEQ ID NO:12) or GGGAGACUGCCAAGAUG (SEQ ID NO:13). The 5′-untranslated region (5′-UTR) of mRNA of this embodiment contains structural elements,Nvhich are recognized by cell-specific RNA-binding proteins, thereby affecting the translation of the molecule. To create recombinant RNA transcripts with short synthetic TRs, the corresponding DNA sequences may be cloned into a plasmid vector upstream of the RBD gene. Table 1 lists the positions of different bases in the mRNA relative to the start codon. T7 promoter (TAATACGACTCACTATA; (SEQ ID NO: 14))) may be combined with the Kozak element consensus sequence upstream of the start codon (ATG). Transcription from T7 promoter begins with the first G after the TATA element. The following six bases after the TATA element (GGGAGA) (SEQ ID NO:15) help provide high yields and homogenous 5′mRNA ends during in vitro transcription. This template-sequence results in an RNA, which has the sequence GGGAGACUGCCA (C/A) (C/G) AUG (SEQ ID NO:16) as its 5′-UTR.

Table I shows two minimal UTRs with best results as 5′-UTRs.

TABLE 1
Sequences of Synthetic 5′-Untranslated Region

| | | Trans- | | | |
|---|---|---|---|---|---|
| Minimal | | cription | Extra | Kozak | Start |
| UTR | Promoter | start site | nucleotides | sequence | codon |
| UTR1 | T7 | GGGAGA | CT | GCCACC | ATG |
| UTR2 | T7 | GGGAGA | CT | GCCAAG | ATG |

In a further embodiment, the mRNA comprises a 3′ untranslated region comprising on or two copies of a beta globin mRNA Any beta globin mRNA 3′-UTR may be used as deemed suitable for an intended purpose. In one embodiment, the beta globin mRNA 3′-UTR comprises the amino acid sequence of SEQ ID NO:18.

(SEQ ID NO: 18)
GCUCGCUUUCUUGCUGUCCAAUUUCUAUUAAAGGUUCCUUUGUUCCCUA
AGUCCAACUACUAAACUGGGGGAUAUUAUGAAGGGCCUUGAGCAUCUGG
AUUCUGCCUAAUAAAAAACAUUUAUUUUCAUUGC

In another embodiment, the mRNA encodes a signal sequence, to facilitate display by APCs. Any suitable signal sequence may be used. In one embodiment, the signal sequence is encoded at the N-terminus of the polypeptide. In non-limiting embodiments, the signal sequence may comprise MMYRMQLLSCIALSLALVTNS (SEQ ID NO: 22) or MYRMQLLSCIALSLALVTNS (SEQ ID NO:23), which may optionally be present at the N-terminus of the encoded polypeptide. In one embodiment, the signal sequence comprises the amino acid sequence of SEQ ID NO: 23.

As noted above, the examples show that there is no loss of immunogenieity multivalent compositions as compared to each monovalent component. Convergence of mutations in the RBD indicates that a small valency can cover a large, number of variants, and thus these multivalent compositions provide a significant clinical benefit. Thus, in another embodiment, the disclosure provides composition comprising a plurality of polypeptides, multimers, scaffolds, nucleic acids, or mRNA nucleic acids according to any embodiment or combination of embodiments disclosed herein. In one embodiment, the compositions comprises a plurality (2, 3, 4, 5, 6, 7, 8, 9, 10, 12, 13, 14, 15, 17, 18, 19, 20, or more) of polypeptides according to any embodiment or combination of embodiments disclosed herein. In another embodiment, the compositions comprise a.

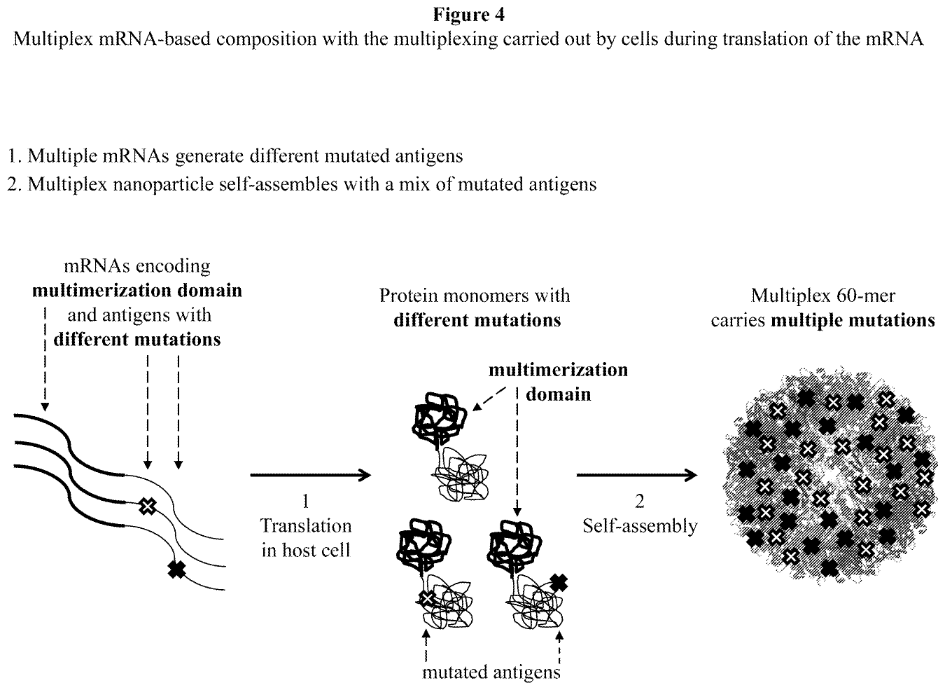

plurality of nucleic acids, such as mRNAs, that encode 2, 3, 4, 5, 6, 7, 8, 9, 10, 11, 12, 13, 14, 15, 17, 18, 19, 20 or more different polypeptides of any embodiment or embodiments disclosed herein. In one embodiment of the compositions disclosed herein., die. compositions comprise two or more (2, 3, 4, 5, 6, 7, 8, 9, 10, 11, 12, 13, 14, 15, or more) polypeptides, multimers, scaffolds, or nucleic acids (such as mRNA) that each comprise or encode a different isolated polypeptide of any embodiment or combination of embodiments disclosed herein. FIG. 4 shows an example of a multiplex mRNA-based composition according to these embodiments, with the multiplexing carried out by cells during translation of the mRNA.

Itr one embodiment, the composition comprises mRNAs that encode 2, 3, 4, or more polypeptides comprising an ammo acid sequence at least 70%, 75%, 80%, 85%, 90%, 91%, 92%, 93%, 94%, 95%, 96%, 97%, 98%, 99%, or 100% identical to the amino acid sequence of any one of SEQ ID NOS:7-10 and 25-32. In another embodiment, the composition comprises mRNAs that encode 2, 3, or 4 polypeptides comprising an amino acid sequence at least 75%, 80%, 85%, 90%, 91%, 92%, 93%, 94%, 95%, 96%, 97%, 98%, 99%, or 100% identical to the amino acid sequence of SEQ ID NOS:29-32. In a still further embodiment, the composition comprises nucleic acids that encode 2, 3, or 4 polypeptides comprising the amino acid sequences selected from SEQ ID NOS:29-32.

In another aspect, the disclosure provides pharmaceutical compositions comprising

(a) the polypeptide, the multimer, the scaffold, the nucleic acid, the composition, the recombinant expression vector, and/or the cell of any embodiment or combination of embodiments herein; and

(b) a pharmaceutically acceptable carrier.

The pharmaceutical compositions of the disclosure may be used, for example, in the methods of the disclosure. In one embodiment the compposition comprises the pharmaceutically acceptable carrier and 1, 2, 3, 4, 5, 6, 7, 8, 9, 10, 11, 12, 13, 14, or all 15 of.

(a) the isolated polypeptide of SEQ ID NO:5;

(b) the isolated polypeptide of SEQ ID NO:6;

(c) the isolated polypeptide of SEQ ID NO:7;

(d) the isolated polypeptide of SEQ ID NO:8;

(e) the isolated polypeptide of SEQ ID NO:9;

(f) the isolated polypeptide of SEQ ID NO:10;

(g) the isolated polypeptide of SEQ ID NO:24;

(h) the isolated polypeptide of SEQ ID NO:25,

(i) the isolated polypeptide of SEQ ID NO:26;

(j) the isolated polypeptide of SEQ ID NO:27

(k) the isolated polypeptide of SEQ ID NO:28;

(l) the isolated polypeptide of SEQ ID NO:29;

(m) the isolated polypeptide of SEQ ID NO:30;

(n) the isolated polypeptide of SEQ ID NO:31; and/or

(o) the isolated polypeptide of SEQ ID NO:325;

or multimers or scaffolds thereof.

one embodiment, the pharmaceutical composition comprises the isolated polypeptide of 1, 2, 3, 4, 5, 6, 7, 8, 9, 10, 11, or all 12 of SEQ ID NOS:7-10 and 25-32; or multimers or scaffolds thereof In a further embodiment, the pharmaceutical composition comprises the isolated polypeptide of 1, 2, 3, or 4, of SEQ ID NOS:29-32; or inuitiiners or scaffolds thereof

In another embodiment, compositions comprise

(a) the mRNA or composition of any embodiment or combination of embodiments herein; and

(b) a cationic lipid carrier, such as a liposome, or a cationic protein, such as protamine.

Any cationic lipid carrier may be used as deemed appropriate for an intended use, including but not limited to liposomes. Alternatively, any cationic protein may be used, including, but not limited to protamine.

In one embodiment, the mRNAs present in the pharmaceutical composition encode polypeptides comprising the amino acid sequence of 1, 2, 3, 4, 5, 6, 7, 8, 9, 10, 11, 12, 13, 14, or all 15 of:

(a) the isolated polypeptide of SEQ ID NO:5;

(b) the isolated polypeptide of SEQ ID NO:6;

(c) the isolated polypeptide of SEQ ID NO:7;

(d) the isolated polypeptide of SEQ ID NO:8;

(e) the isolated polypeptide of SEQ ID NO:9;

(f) the isolated polypeptide of SEQ ID NO:10;

(g) the isolated polypeptide of SEQ ID NO:24;

(h) the isolated polypeptide of SEQ ID NO:25;

(i) the isolated polypeptide of SEQ ID NO:26

(j) the isolated polypeptide of SEQ ID NO:27

(k) the isolated polypeptide of SEQ ID NO:28;

(l) the isolated polypeptide of SEQ ID NO:29;

(m) the isolated polypeptide of SEQ ID NO:30;

(n) the isolated polypeptide of SEQ ID NO:31; and/or (o) the isolated polypeptide of SEQ ID NO:32.

In another embodiment, the mRNAs present in the pharmaceutical composition encode polypeptides comprising the arnino acid sequence of 1, 2, 3, 4, 5, 6, 7, 8, 9, 10, 11, or all 12 of SEQ ID NOS:7-10 and 25-32.

In a further embodiment, the mRN,As present in the pharmaceutical composition encode polypeptides comprising the ammo acid sequence of 1, 2, 3, or all 4 of SEQ ID NOS:29-32.

The pharmaceutical composition may further comprise (a) a lyoprotectant; (b) a surfactant, (c) a bulking agent; (d) a tonicity adjusting agent; (e) a stabilizer; (f) a preservative andlor (g) a buffer.

In some embodiments, the buffer in the pharmaceutical composition is a Tris buffer, a histidine buffer, a phospluite buffer, a citrate buffer or an acectate buffer. The pharmaceutical composition may also include a lyoproteetant, e.g. sucrose, sorbitol or trehalose, certain embodiments, the pharmaceutical composition includes a preservative e.g. benzalkonium chloride, benzethonium, chlorohexidine, phenol, in-cresol, benzyl alcohol, methylparaben, propylparaben, chlorobutanol, o-cresol: p-crestal, chlorocresol, phenylmercuric nitrate: thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sarbitan monolaurate, sorbitan monopalmitate, sorbitan monostearate, sorbitan monooleatc, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, l cine, methionine, mannitol, dextrose, inositol, sodium chloride, arginine and arginine hydrochloride, in other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptide, the multimer, the seaffbld, the nucleic acid, the composition, the recombinant expression vector, and/or the cell of any embodiment or combination of embodiments herein may be the sole active agent in the pharmaceutical composition, or the composition may further comprise one or more other active agents suitable for an intended use.

In another aspect, the disclosure provides methods for treating a SARS coronavirus infection, comprising administering to a subiect infected with a SARS coronavirus an amount effective to treat the infection of the polypeptide, the multimer, the scaffold, the nucleic acid, the composition, the recombinant expression vector, the cell, and/or the pharmaceutical composition of any claim herein.

As used herein, “treat” or “treating” means accomplishina one or more of the following in an individual that already has a SARS coronavirus infection: (a) reducing the severity of the infection; (b) limiting or preventing, developmetat of symptoms characteristic of the infection being treated; (c) inhibiting vvorsening of symptoms characteristic of the infection, and (d) limiting or preventing recurrence of symptoms in patients that were previously symptomatic for the infection.

In another aspect, the disclosure provides methods for limiting development of a SARS coronavirus infection, comprising administering to a subject at risk of SARS coronavirus infection an amount effective to limit development of a SARS coronavirus infection of the polypeptide, the multimer, the scaffold, the nucleic acid, the composition, the recombinant expression vector, the cell, and/or the pharmaceutical composition of any claim herein.

As used herein, “limiting” or “limiting development of” means accomplishing one or more of the following in an individual that does not have a SARS coronavirus infection: (a) preventing infection: (b) reducing the severity a subsequent infection; and (c) limiting or preventing development of symptoms after a subsequent infection.

In a further aspect, the disclosure provides methods for generating an immune response in a subject, comprising administering to the subject an amount effective to generate an immune response of the polypeptide, the multimer, the scaffold, the nucleic acid, the composition, the recombinant expression vector, the cell, and/or the pharmaceutical composition of any claim herein.

In this aspect, generating an immune response can be used to prevent infection, treat an existing infection or limit development of a subsequent infection.

In all of the above aspects, an “amount effective” refers to an amount of the therapeutic that: is effective for treating and/or limiting the infection. The polypeptide, the multimer, the scaffold, the nucleic acid, the composition, the recombinant expression vector, the cell, andior the pharmaceutical composition may be administered by any suitable route. In one embodiment of all of these aspect, the polypeptide, the multimer, the scaffold, the nucleic acid, the composition, the recombinant expression vector, the cell, andfor the pharmaceutical composition may be administered by subcutaneous, intradermal or intramuscular injection. In another embodiment, the method comprises administering to the subject an effective amount of the pharmaceutical composition with a needle-free injection system.

The subject in any of the methods disclosed herein may be any subject infected with or at risk or a SARS coronavirus infection, including but not limited to a human subject.

In another aspect, the disclosure provides methods for monitoring a SARS coronavirus induce disease in a subject and/or monitoring response of the subject to immunization by a SARS coronavirus vaccine, comprising contacting the polypeptide, the multimer, the scaffold, and/or the pharmaceutical composition of any claim herein with a bodily fluid from the subject and detecting SARS coronavirus-binding antibodies in the bodily fluid of the subject. In this embodiment, a change in SARS coronavirus-binding antibodies in the bodily fluid of the subject can be monitored over time after the therapeutic or prophylactic methods disclosed herein, or any other therapeutic or prophylactic methods to treat or limit development of a SARS coronavirus-induced disease.

In one embodiment, the bodily fluid comprises serum or whole blood.

In a further aspect, the disclosure provides methods for detecting SARS coronavirus binding antibodies, comprising

(a) contacting the polypeptide, the multimer, the scaffold and/or the pharmaceutical composition of any claim herein kvith a composition comprising a candidate SARS coronavirus binding antibody under conditions suitable for binding of SARS coronavirus antibodies to the polypeptide, the multimer: the scaffold, and/or the pharmaceutical composition, and

(b) detecting SARS coronavirus antibody complexes with the polypeptide, the multimer, the scaffold, andior the phannaceutical composition. In this embodiment, the reagents disclosed, herein can be used in testing a subject for SARS coronavirus infection.

In one embodiment, the method further comprises isolating the SARS eoronavirus antibodies that can be used, for example, as therapeutic antibodies to treat a subject having a SARS coronavirus infection.

In a further embodiment, the disclosure provides methods for producing SARS coronavirus antibodies, comprising

(a) administering to a subject an amount effective to generate an antibody response of the polpeptide, the multimer, the scaffold, the nucleic acid, the composition, the recombinant expression vector, the cell, and/or the pharmaceutical composition of any claim herein; and

(b) isolating antibodies produced by the subject. In this aspect, antibodies may be isolated and used, for example, as therapeutic antibodies to treat a subject having SARS coronavirus infection.

### EXAMPLES

### Example 1

### Immunogenicity in Mouse of VX3025r mRNA Vaccine

The following experiments confirm that the mRNA vaccine VX3025r encoding for the amino acid sequence of SEQ 1D NO:10 elicits neutralizing antibodies in vaccinated mouse with inhibition of the RBD-ACE2 interaction comparable or superior to COVID-19 human patient sera.

Construct in pUC19 Plasmid

A construct with the 5′ minimal untranslated region UTR1 of Table 1, the human 11,-2 signal sequence, a nucleotide sequence encoding the amino acid sequence of SEQ ID NO:10 and an N-terminal signal sequence (MMYRMQLLSCIALSLALVTNS; SEQ ID NO 22), the 3′UTR region of SEQ ID NO:18 a poly(A) tail of 70 adenosine residues and the BsmBI restriction site was cloned into the pUC19 vector.

mRNA Transcription and Capping

The supereoiled pUC19 DNA was upsealed and linearized with the enzyme BsmBI, and in vitro transcription was performed with 17 polymerase in a 2 mL reaction. The mRNA was capped on the 5′ end with vaecinia enzymatic capping. Final yield of VX3025r mRNA was 6.0 mg after purification.

Vaccine Formulation

The mRNA VX3025r was complexed with the polycationic protein protamine by addition of protamine to the mRNA at a mass ratio of 1:5. The VX3025r vaccine was prepared on each injection day with final VX3025r mRNA concentration of 840 μg/mL.

Immunization

A first group of N-4 CB6F1/J female mice (The Jackson Laboratory) 6-8 weeks old was dosed by intradermal injection at the ear pinna under 1-5% isoflurane anesthesia with the vaccine at week 0 and 2. Dose 42 μg/50 μL.

A second group of N=4 CB6F1/J female mice 6-8 weeks old was dosed by intramuscular injection at the caudal thigh with a. needle free injection system (Tropis injector modified for mouse injection, Pharrnalet) under 1-5% isoflurane anesthesia at week 0 and 2. Dose 42 μg/50 μL.

Blood Collection

Blood was collected into clot activator tubes via retro-orbital capillary tube collection under 1-5% isoflurane anesthesia at 200 μL per collection in week 0 (prior to dose), 2 (prior to second dose) and 4. All blood samples were allowed to clot at room temperature, centrifuged 10 ambient (20° C.T) at 3000 RPM for 15 minutes, and serum supernatant was stored frozen at −80° C.

ELISA Analysis

To determine it VX3025r elicits neutralizing antibodies we analyzed randomly selected samples of week 0 collection and all samples of week 4 collection with the SARS-CoV-2

surrogate Virus Neutralization Test (sVNT) Kit (CienScript). The assay detects any antibodies in serum and plasma that neutralize the RBD-ACE2 interaction. The test is both species and isotype independent.

The SARS-CoV-2 sVNT kit is a blocking ELISA detection tool, which mimics the virus neuttralization process. The kit contains two key components: the Horseradish peroxidase (HRP) conjugated recombinant SARS-CoV-2 RBD fragment (HRP-RBM and the human ACE2 receptor protein (hACE2). The protein-protein interaction between HRP-RBD and hACE2 is blocked by neutralizing antibodies against SARS-CoV-2 RBD.

First, the samples and controls are pre-incubated with the HRP-RBD to allow the binding of the circulating neutralization antibodies to HRP-RBD. The mixture is then added to the capture plate which is pre-coated with the human ACE2 protein. The unbound HRP-RBD as well as any HRP-RBD bound to non-neutralizing antibody is captured on the plate, while the circulating neutralization antitiodies_HRP-RBD complexes remain in the supernatant and get removed during washing. After washing steps, 3, 3′, 5, 5′-tetramethylbenzidine (TMB) solution is added, making the color blue. By adding Stop Solution, the reaction is quenched and the color turns yellow. This final solution is read at 450 nm in a microtiter plate reader. The absorbance of the sample is in dependent on the titer of the anti-SARS-CoV-2 neutralizing antibodies.

The RBD-ACE2 interaction inhibition rate is calculated with the net optical density (OD450) of sample and kit negative control as follows:

Inhibition=(1−OD value of sample/OD value of negative control)×100%

The positive and negative cutoff for SARS-CoV-2 neutralizing antibody detection is used for interpretation of the inhibition rate. The cutoff value of 20% is based on validation with a panel of confirmed COVID-19 patient sera and healthy control sera (GenScript).

Results

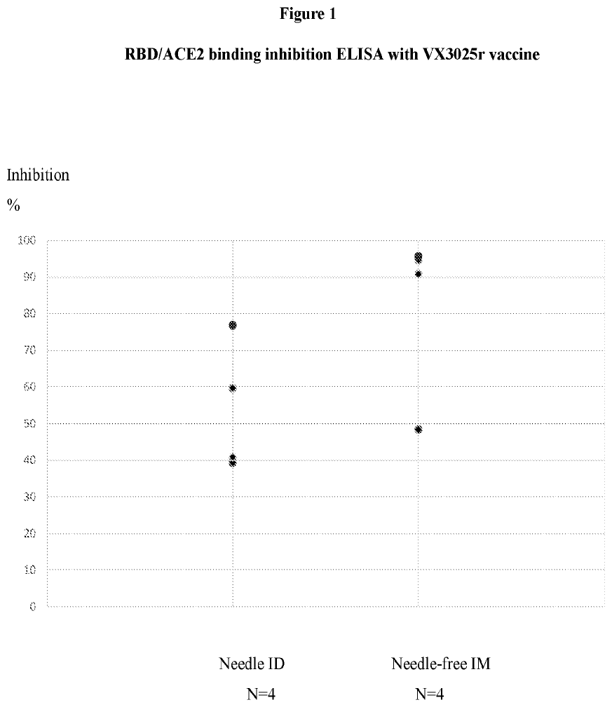

In the first group for all 4 week 4 samples inhibition percentage was higher than 20% indicating detection of SARS-CoOV-2 neutralizing antibodies in the mouse sera (mean 54.16, standard deviation 17.72, range 3932 to 76.81) (FIG. 1)

In the second group for all 4 week 4 samples inhibition percentage was higher than 20% indicating detection of SARS-CoV-2 neutralizing antibodies in the mouse sera (mean 82.36, standard deviation 22.76, range 48.36 to 95.64) (FIG. 1). The inhibition rate with needle-free intramuscular injection was much higher with a mean value of 82.36% as compared to a mean value of 54.16% with the standard ear pinna intradermal injection. Moreover for ¾ mouse samples inhibition ratio was higher than 90% (range 90.83% to 95.64 suggesting strong neutralization activity (FIG. 1).

For the two groups the inhibition of random week 0 samples ranged from 7.98% to 9.15% with a mean value of 8.57% indicating no detectable SARS-CoV-2 neutralizing antibody.

Therefore the VX3025r schedule induced neutralizing antibodies in all mouse sera with inhibition of RBD-ACE2 interaction comparable to human COVID-19 patient sera, and superior neutralization with the needle-free intramuscular route of administration,

### Example 2

### Immunogenicity in Mouse of VX3025r mRNA Vaccine

To confirm the results of the second group of Example 1 the sera of all mice with RBD-ACE2 interaction inhibition ratio >90% at week 4 was tested for neutralization of authentic wild type SARS-CoV-2 virus.

Blood Collection

Blood was collected into clot activator tubes via retro-orbital capillary tube collection under 1-5% isoflurane anesthesia, at 200 μL per collection in week 0 (prior to dose), 2 (prior to second dose), 4, 6, and terminal cardiac puncture collection in week 12. All blood samples were allowed to clot at room temperature, centrifuged ambient: (20° C.) at 3000 RPM for 15 minutes, and serum supernatant was stored frozen at −80° C.

SARS-CoV-2 Neutralization Assay

Sera from 3 mice collected at week 2.4, 6 and 12 were tested for SARS-CoV-2 neutralization. The serial dilutions of heat-inactivated (30 min at 56° C.) mouse sera were prepared in quadruplicates in 96-well cell culture plates using Dulbecco's Modified Eagle Medium (DMEM) cell culture medium (50 well). To each well, 50 μL of DME containing 100 tissue culture infectious dose 50% (TCID50) of SAVRS-CoV-2 were added and incubated for 60 min at 37° C. Subsequently, 100 μL of Vero E6 cell suspension (100,000 cells/mL in DMEM with 10% fetal bovine serum) were added to each well and incubated for 72 hat 37° C. The cells were fixed for 1 h at mom temperature with 4% buffered formalin solution containing 1% crystal violet. Finally, the microtifer plates were rinsed with deionized water and immune serum-mediated protection from cytopathic effect was visually assessed. Neutralization doses 50% (ND50) values were calculated according to the Spearman and Kärber method.

Results

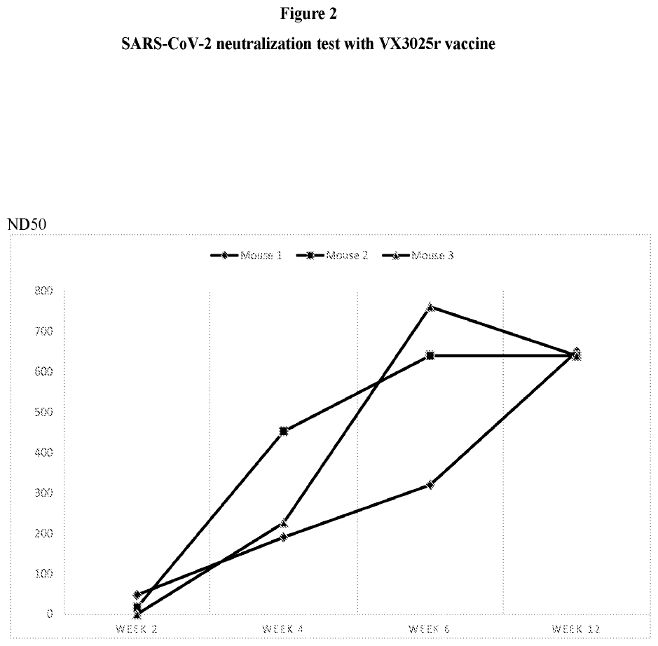

SARS-Cov-2 neutralization by, the three mouse sera showed regular progression at week 2, 4, 6 and 12 with ND50 reaching 640 or above at week 12 for all mice as shown in Table 2 and FIG. 2.

TABLE 2
ND50 values of VX3025r elicited mouse sera

| ND50 value | Week 2 | Week 4 | Weck 6 | Week 12 |
|---|---|---|---|---|
| Mouse 1 | 48 | 190 | 320 | 650 |
| Mouse 2 | 17 | 453 | 640 | 640 |
| Mouse 3 | 0 | 226 | 761 | 640 |

Therefore the VX3025r needle-free intramuscular vaccination schedule induced neutralizing antibodies in mouse sera starting from 2 weeks after the first dose and reaching neutralization dose 50% of 640 or above 12 weeks after the first dose car 10 weeks after the second dose.

### Example 3

### Immunogenicity in Mouse of Bivalent mRNA

The following experiments confirm that a bivalent mRNA vaccine encoding for the two different amino acid sequences of SEQ ID NO:10 and SEQ ID NO:29 elicits neutralizing antibodies in vaccinated mouse sera, with inhibition of the RBD wild type-ACE2 interaction or the RBD alpha variant-ACE2 interaction comparable or superior to human sera of patients infected with the SARS-CoV-2 wild type or the SARS-CoV-2 alpha variant.

Constructs in pUC19 Plasmid

Two constructs with the 5′ minimal untranslated ration UTR1 of Table 1, the human IL-2 signal sequence, a nucleotide sequence encoding the ammo acid sequence of SEQ ID NO:10 with or without the mutation N501Y (residue 171 in SEQ ID NO:1 or 2), the 3suTR region of SEQ ID NO:18, a poly(A) tail of 70 adenosine residues and the BsmBI restriction site were cloned into the pUC19 vector.

mRNA Transcription and Capping

The supercoiled pUC19 DNAS Were upscaled and linearized with the enzyme BsmBI, and in vitro transcription was performed with T7 polymerase in 2 mL reactions. The two mRNAs were capped on the 5′ end with vaccinia enzymatic capping. Final yield of VX3025rD mRNA encoding a polypeptide comprising the ammo acid sequence of SEQ ID NO:10 and the N-terminal signal sequenee of MYRMQLLSCIALSLALVTNS (SEQ ID NO:23) was 6.6 mg after purification and VX3025rM1 mRNA encoding for the alpha variant sequence (N501Y) was 7.44 mg after purification.

Vaccine Formulation

The two mRNAs VX3025rD and VX3025rM1 were complexed with the polycationic protein prolamine by addition of protamine to the mRNA at a mass ratio of 1:5. Specifically, VX3025rD protamine were mixed in a 1:5 mass ratio and (separately) VX3025IM1+ protamine were mixed in a 1:5 mass ratio to produce (separately) two monovalent vaccine complexes. The two separate monovalent vaccine complexes (VX3025rD and VX3025rM1) were mixed in 1:1 mass ratio to produce the bivalent vaccine complex. The two monovalent vaccines VX3025rD and VX3025rM1 and the bivalent vaccine VX3025rB1 were prepared on each injection day with final total mRNA concentration of 840 μg/mL.

Immunization

3 groups of N=6 CB6F1/J female mice 6-8 weeks old were dosed by intramuscular injection at the caudal thigh with a needle-free injection system (Tropic in modified for mouse injection, PharmaJet) under isoflurane anesthesia at week 0 and 3. Dose 42 μg/50 μL. The first group was dosed with VX3025rD, the second group with VX3025rM1 and the third group with the bivalent vaccine VX3025rB1.

Blood Collection

Blood was collected into clot activator tubes via retro-orbital capillaiy tube under 1-5% isoflurane anesthesia at 200 μL, per collection in week 0 (prior to dose), 3 (prior to second dose) and 6. All blood samples were allowed to clot at room temperature, centrifuged ambient (20° C.) at 3000 RPM for 15 minutes, and serum supernatant was stored frozen at −80° C.

HSA Analysis

To deterrmine if VX3025r elicits neutralizing antibodies we analyzed randomly selected samples of week 0 collection and all samples of week 5 collection with the SARS-CoV-2 surrogate Virus Neutralization lest (sVNT) Kit (GenSeript) described in Example 1. The assay detects any antibodies in serum that neutralize the RBD-ACE2 interaction. In this experiment two different assays were perfortmed with two different Horseradish peroxidase (HRP) conjugated recombinant SARS-CoV-2 RBD fragments (HRP-RBD). The first HRP-RBD_{wt} contains the wild type RBD amino acid sequence, the second HRP-RBDₐₗₚₕₐ contains the mutation N501Y of the SARS-CoV-2 alpha variant. The protein-protein interaction between HRP-RBD_{wt} or HRP-RBDₐₗₚₕₐ and hACE2 is blocked by neutralizing antibodies usainst BARS-CoV-2 RBD_{wt} or RBDₐₗₚₕₐ.

The RBD_{wt}-ACE2 or RBDₐₗₚₕₐ-ACE2 interaction inhibition rate is calculated with the net optical density (OD450) of sample and kit negative control as follows:

Inhibition=(1−OD value of sample/OD value of negative control)×100%

The positive and negative cutoff for SARS-CoV-2 neutralizing, antibody detection is used for interpretation of the inhibition rate. The cutoff value of 20% is based on validation with a panel of confirmed COVID-19 patient sera and healthy control sera (GenScript).

The sera of the first group immunized with VX3025rD was tested with RBD_{wt}, the sera of the second group immunized with VX3025rM1 was tested with RBDₐₗₚₕₐ, and the sera of the third group immunized with the bivalent VX3025rB1 was tested with RBD_{wt}.

Results

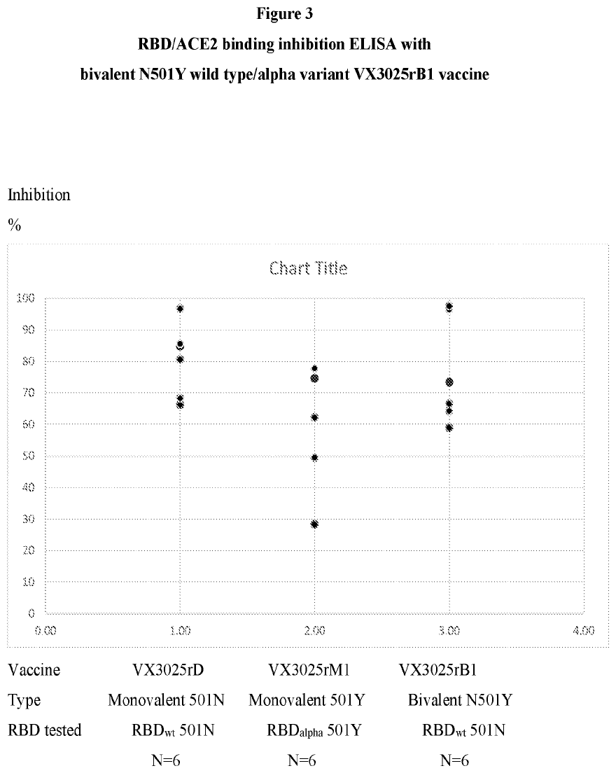

In the first group receiving the monovalentt wild type vaccine for all 6 week 6 samples inhibition percentage of wild type RBD intoaction was higher than 20% indicating detection of SARS-CoV-2 wild type neutralizing antibocaes in the mouse sent (mean 80.34, standard. deviation 11.46, range 66.27 to 96.62) (FIG. 3).

In the second group receiving the monovalent alpha variant vaccine for all 6 week 6 samples inhibition percentage of alpha variant RBD interaction was higher than indicating detection of SARS-CoV-2 alpha variant neutralizing antibodies in the mouse, sera (mean 5.3.37, standard deviation 21.93, range 28.01 to 77.72) (FIG. 3).

In the third group receiving the bivalent vaccine for all 6 week 6 samples inhibition percentage of wild type RBD interaction was higher than 20% indicating detection of SARS-CoV-2 wild type neutralizing antibodies in the mouse sera (mean 76.15, standard deviation 16.74, range 58.91 to 97.49) (FIG. 3).

For the first group the inhibition of random week 0 samples ranged from 4. 77% to 7.21% with a mean value of 5.87% indicating no detectable SARS-CoV-2 neutralizing antibody. For the second group the inhibition of random week 0 samples ranged from 3.32% to 7.12% with a mean value of 5.87% indicating no detectable SARS-CoV-2 neutralizing antibody. For the third group the inhibition of random week 0 samples ranged from 1.85% to 8.04% with a mean value of 5.38% indicating no detectable SARS-CoV-2 neutralizing antibody.

Therefore the vaccination schedule with the monovalent vaccines induced neutralizing antibodies with inhibition of RBD-ACE2 interaction comparable or superior to sent of human patients infected with the wild type virus or the alpha variant, and the vaccination schedule with the bivalent vaccine induced neutralizing antibodies with inhibition of RBD-ACE2 interaction comparable to the inhibition with the monovalent wild type vaccine.

### Example 4

### Immunogenicity in Mouse of Bivalent mRNA Vaccine Encoding for Amino Acid Sequences of SEQ ID NO:10 and SEQ ID NO:29

SARS-CoV-2 Neutralization Assay

To confirm the results of the experiment of Example 3 the sera of all mice immunized with the bivalent VX3025rB1 vaccine and with RBD-ACE2 interaction inhibition ratio >90% at week 6 were tested for neutralization of authentic SARS-CoV-2 wild type and alpha variant with the SARS-CoV-2 neutralization assay described in Example 2 in triplicates.

Results

TABLE 3
ND50 values of VX3025rB1 elicited mouse sera

| | RBD-ACE2 | ND50 value | ND50 value |
|---|---|---|---|
| | Inhibition ratio | Wild type | Alpha variant |
| Mouse 1 | 96.34% | 160 | 640 |
| Mouse 2 | 97.49% | 640 | 640 |

Therefore, the bivalent VX3025rB1 needle-free intramuscular vaccination schedule induced neutralizing antibodies in the sera of these mice 6 weeks aftr the first dose and 3 weeks after the second dose, against both SARS-CoV-2 and the SARS-CoV-2 alpha variant.

1. A polypeptide comprising:
(a) a receptor binding domain (RBD) comprising an amino acid sequence at least 70%, 75%, 80%, 85%, 90%, 91%, 92%, 93%, 94%, 95%, 96%, 97%, 98%, 99%, or 100% identical to the amino acid sequence of any one of SEQ ID NOS:1-2 or 11; and
(b) a multimerization domain capable of generating multimers comprising at least 60 copies of the polypeptide.

2-4. (canceled)

5. The polypeptide of claim 1, wherein the multimerization domain comprises an amino acid sequence at least 70%, 75%, 80%, 85%, 90%, 91%, 92%, 93%, 94%, 95%, 96%, 97%, 98%, 99%, or 100% identical to the amino acid sequence of SEQ ID NO: 3-4.

6-11. (canceled)

12. A multimer comprising 60 or more copies of a receptor binding domain (RBD) comprising an amino acid sequence at least 70%, 75%, 80%, 85%, 90%, 91%, 92%, 93%, 94%, 95%, 96%, 97%, 98%, 99%, or 100% identical to the amino acid sequence of any one of SEQ ID NOS:1-2 or 11.

13-15. (canceled)

16. A multimer, comprising 60 or more copies of the polypeptide of claim 1.

17-25. (canceled)

26. A nucleic acid encoding the polypeptide of claim 1.

27. A recombinant expression vector comprising the nucleic acid of claim 26 operatively linked to a suitable control sequence.

28. A recombinant host cell comprising the recombinant expression vector of claim 27.

29. The nucleic acid of claim 26 wherein the nucleic acid comprises mRNA.

30. The nucleic acid of claim 29, wherein the mRNA comprises a 5′ cap.

31. The nucleic acid of claim 29, further comprising a poly(A) tail of between 50 and 120 contiguous adenosine residues.

32. The nucleic acid of claim 29, wherein the mRNA comprises a 5′ untranslated region comprising the nucleic acid sequence of SEQ ID NO:12 or 13.

33. The nucleic acid of claim 29, wherein the mRNA comprises a 3′ untranslated region comprising one or two copies of a beta globin mRNA 3′ -UTR.

34. The nucleic acid of claim 33, wherein the beta globin mRNA 3′-UTR comprises the nucleic acid sequence of SEQ ID NO:18.

35. The nucleic acid of claim 29, wherein the mRNA encodes a signal sequence, optionally wherein the signal sequence is at the N-terminus of the encoded polypeptide, and optionally wherein the signal sequence comprises the amino acid sequence of SEQ ID NO:22 or 23.

36. The nucleic acid of claim 35, wherein the signal sequence comprises the amino acid sequence of SEQ ID NO: 23.

37. (canceled)

38. A composition of claim 37, comprising nucleic acids that encode 2 or more polypeptides comprising an amino acid sequence at least 70%, identical to the amino acid sequence of any one of SEQ ID NOS:7-10 and 25-32.

39. (canceled)

40. The composition of claim 38, comprising nucleic acids, such as mRNA, that encode 2, 3, or 4 polypeptides comprising the amino acid sequences selected from SEQ ID NOS:29-32.

41-44. (canceled)

45. A pharmaceutical composition comprising:
(a) the nucleic acid of claim 29; and
(b) a cationic lipid carrier or a cationic protein.

46. (canceled)

47. The pharmaceutical composition of claim 45, wherein the nucleic acids present in the pharmaceutical composition encode polypeptides comprising the amino acid sequence of 1 or more of:
(a) the polypeptide of SEQ ID NO:5;
(b) the polypeptide of SEQ ID NO:6;
(c) the polypeptide of SEQ ID NO:7;
(d) the polypeptide of SEQ ID NO:8;
(e) the polypeptide of SEQ ID NO:9;
(f) the polypeptide of SEQ ID NO:10;
(g) the polypeptide of SEQ ID NO:24;
(h) the polypeptide of SEQ ID NO:25;
(i) the polypeptide of SEQ ID NO:26;
(j) the polypeptide of SEQ ID NO:27
(k) the polypeptide of SEQ ID NO:28;
(l) the polypeptide of SEQ ID NO:29;
(m) the polypeptide of SEQ ID NO:30;
(n) the polypeptide of SEQ ID NO:31; and/or
(o) the polypeptide of SEQ ID NO:32.

48-49. (canceled)

50. A method for
(a) treating a SARS coronavirus infection, comprising administering to a subject infected with a SARS coronavirus an amount effective to treat the infection of the polypeptide of claim 1; or
(b) limiting development of a SARS coronavirus infection, comprising administering to a subject at risk of SARS coronavirus infection an amount effective to limit development of a SARS coronavirus infection of the polypeptide of claim 1; or
(c) generating an immune response in a subject, comprising administering to the subject an amount effective to generate an immune response of the polypeptide of claim 1; or
(d) monitoring a SARS coronavirus-induced disease in a subject and/or monitoring response of the subject to immunization by a SARS coronavirus vaccine, comprising contacting the polypeptide of claim 1 with a bodily fluid from the subject and detecting SARS coronavirus-binding antibodies in the bodily fluid of the subject or
(e) detecting SARS coronavirus binding antibodies, comprising
(i) contacting the polypeptide of claim 1 with a composition comprising a candidate SARS coronavirus binding antibody under conditions suitable for binding of SARS coronavirus antibodies to the polypeptide, and
(b) detecting SARS coronavirus antibody complexes with the polypeptide, or
(f) producing SARS coronavirus antibodies, comprising
(a) administering to a subject an amount effective to generate an antibody response of the polypeptide of claim 1, and
(b) isolating antibodies produced by the subject.

51-59. (canceled)
